SEQUENCE LISTING (40 sequences)

SEQ ID NO: 1 - protein, 9 aa, Artificial sequence
  Synthetic peptide
GGGGRGDSP

SEQ ID NO: 2 - protein, 13 aa, Artificial sequence
  Synthetic peptide
RLVSYNGIIFFLK

SEQ ID NO: 3 - DNA, 22 bp, Artificial sequence
  Synthetic oligonucleotide
ctttgtcaagctcatttcctgg

SEQ ID NO: 4 - DNA, 19 bp, Artificial Sequence
  Synthetic oligonucleotide
tcttgctcagtgtccttgc

SEQ ID NO: 5 - DNA, 22 bp, Artificial Sequence
  Synthetic oligonucleotide
ctccaaaagctcaacaccaatg

SEQ ID NO: 6 - DNA, 19 bp, Artificial Sequence
  Synthetic oligonucleotide
atttgtccatctccagccg

SEQ ID NO: 7 - DNA, 23 bp, Artificial Sequence
  Synthetic oligonucleotide
gctattaaagtgacagtggacgg

SEQ ID NO: 8 - DNA, 22 bp, Artificial Sequence
  Synthetic oligonucleotide
ggcgatcagagaacaaactagg

SEQ ID NO: 9 - DNA, 21 bp, Artificial Sequence
  Synthetic oligonucleotide
tgttatgggtgaaactctggg

SEQ ID NO: 10 - DNA, 20 bp, Artificial Sequence
  Synthetic oligonucleotide
agagctgattccgaagttgg

SEQ ID NO: 11 - DNA, 20 bp, Artificial Sequence
  Synthetic oligonucleotide
ccagttaaacttgacgctgc

SEQ ID NO: 12 - DNA, 21 bp, Artificial Sequence
  Synthetic oligonucleotide
gaaactgtggatgtctctggg

SEQ ID NO: 13 - DNA, 22 bp, Artificial Sequence
  Synthetic oligonucleotide
accaagaacttccgattatccc

SEQ ID NO: 14 - DNA, 22 bp, Artificial Sequence
  Synthetic oligonucleotide
cagtaccagtgtcagtatcagc

SEQ ID NO: 15 - DNA, 21 bp, Artificial Sequence
  Synthetic oligonucleotide
ttgatgccattaccagtctcc

SEQ ID NO: 16 - DNA, 20 bp, Artificial Sequence
  Synthetic oligonucleotide
acatggttgggaagttctgg

SEQ ID NO: 17 - DNA, 21 bp, Artificial Sequence
  Synthetic oligonucleotide
gtgaagaggaagacagcacag

SEQ ID NO: 18 - DNA, 21 bp, Artificial Sequence
  Synthetic oligonucleotide
gcccattccaaccattactcc

SEQ ID NO: 19 - DNA, 20 bp, Artificial Sequence
  Synthetic oligonucleotide
accttctacaatgagctgcg

SEQ ID NO: 20 - DNA, 20 bp, Artificial Sequence
  Synthetic oligonucleotide
ctggatggctacgtacatgg

SEQ ID NO: 21 - DNA, 20 bp, Artificial Sequence
  Synthetic oligonucleotide
cctgagtggctgtcttttga

SEQ ID NO: 22 - DNA, 23 bp, Artificial Sequence
  Synthetic oligonucleotide
cgtggagtttgttatctttgctg

SEQ ID NO: 23 - DNA, 19 bp, Artificial Sequence
  Synthetic oligonucleotide
acatcgctcagacaccatg

SEQ ID NO: 24 - DNA, 22 bp, Artificial Sequence
  Synthetic oligonucleotide
tgtagttgaggtcaatgaaggg

SEQ ID NO: 25 - DNA, 20 bp, Artificial Sequence
  Synthetic oligonucleotide
gcacaaatcccttctacccg

SEQ ID NO: 26 - DNA, 22 bp, Artificial Sequence
  Synthetic oligonucleotide
tgaacagcccagtacttattcc

SEQ ID NO: 27 - DNA, 22 bp, Artificial Sequence
  Synthetic oligonucleotide
acccatttacacctacaccaag

SEQ ID NO: 28 - DNA, 21 bp, Artificial Sequence
  Synthetic oligonucleotide
tgtttgcagatctcaggagtg

SEQ ID NO: 29 - DNA, 22 bp, Artificial Sequence
  Synthetic oligonucleotide
gatgacgatgtacaagggatcc

SEQ ID NO: 30 - DNA, 20 bp, Artificial Sequence
  Synthetic oligonucleotide
actggtaatggcatcaaggg

SEQ ID NO: 31 - protein, 4 aa, Artificial Sequence
  Synthetic peptide
RGDS

SEQ ID NO: 32 - protein, 5 aa, Artificial Sequence
  Synthetic peptide
RGDSP

SEQ ID NO: 33 - protein, 6 aa, Artificial Sequence
  Synthetic peptide
RGDSPK

SEQ ID NO: 34 - protein, 5 aa, Artificial sequence
  Synthetic peptide
RGDTP

SEQ ID NO: 35 - protein, 10 aa, Artificial Sequence
  Synthetic peptide
RGDSPASSKP

SEQ ID NO: 36 - protein, 6 aa, Artificial Sequence
  Synthetic peptide
KQAGDV

SEQ ID NO: 37 - protein, 5 aa, Artificial Sequence
  Synthetic peptide
PHSRN

SEQ ID NO: 38 - protein, 5 aa, Artificial Sequence
  Synthetic peptide
YIGSR

SEQ ID NO: 39 - protein, 4 aa, Artificial Sequence
  Synthetic peptide
DGEA

SEQ ID NO: 40 - protein, 4 aa, Artificial Sequence
  Synthetic peptide
VAPG

(19) United States
(12) Patent Application Publication
(10) Pub. No.: US 2023/0330147 A1
(43) Pub. Date: Oct. 19, 2023

(54) ALGINATE-COATED MESENCHYMAL STROMAL AND PROGENITOR CELLS AND METHODS FOR USING THE SAME

(71) Applicant: THE BOARD OF TRUSTEES OF THE UNIVERSITY OF ILLINOIS, Chicago, IL (US)

(72) Inventors: Jae-Won SHIN, Chicago, IL (US); Sing Wan Wong, Chicago, IL (US); Zhangli PENG, Chicago, IL (US)

(21) Appl. No.: 18/043,095

(22) PCT Filed: Aug. 31, 2021
(86) PCT No.: PCT/US2021/048343

§ 371 Date: Feb. 27, 2023

Related U.S. Application Data
(60) Provisional application No. 63/072,282, filed on Aug. 31, 2020.
(60) Provisional application No. 63/120,258, filed on Dec. 2, 2020.

(51) Int. Cl. A61K 35/28; C12N 5/0775; A61P 21/00; A61K 38/18; A61K 47/36; A61P 11/00; A61K 38/19; A61K 9/06; C12N 5/00

(52) U.S. Cl. CPC A61K 35/28; C12N 5/0663; A61P 21/00; A61K 38/1875; A61K 47/36; A61P 11/00; A61K 38/191; A61K 9/06; C12N 5/0012; C12N 2537/10; C12N 2533/74

(57) ABSTRACT

This invention provides a cross-linked alginate hydrogel layer encapsulating single mesenchymal stromal cells or progenitor cells, as well as compositions including the encapsulated cells and to methods of using the same to promote osteogenesis and fibrotic tissue repair in a subject.

This application claims the benefit of priority from U.S. Provisional Application Ser. Nos. 63/072,282, filed Aug. 31, 2020, and 63/120,258, filed Dec. 2, 2020, the contents of which are incorporated herein by reference in their entireties.

This invention was made with government support under grant nos. HL141255, HL125884, GM141147, and AR079561 awarded by the National Institutes of Health and grant no. 1948347 awarded by the National Science Foundation. The government has certain rights in this invention.

### INTRODUCTION

### Background

Cells use tactile mechanisms to physically probe the extracellular matrix (Buxboim & Discher (2010) Nat. Meth. 7:695). Advances in the design of engineered hydrogels have revealed that various matrix biophysical properties are sufficient to impact cellular functions independently of changes in biochemical cues, including matrix elasticity (Engler, et al. (2006) Cell 126:677; Huebsch, et al. (2010) Nat. Mat. 9:518), degradation (Khetan, et al. (2013) Nat. Mat. 12:458), and stress relaxation (Chaudhuri, et al. (Nat. Mat. 15:326). As a result, cells exert traction forces on matrices (Fu, et al. (2010) Nat. Meth. 7:733; Legant, et al. (2010) Nat. Meth. 7:969), and subsequently tune their volume (Guo, et al. (2017) Proc. Natl. Acad. Sci. USA 114:E8618; Lee, et al. (2019) Nat. Comm. 10:529), membrane and intracellular tension (Fischer, et al. (2012) Nat. Protoc. 7:2056; Scarcelli, et al. (2015) Nat. Meth. 12:1132). These physical changes affect downstream biological functions, such as stem cell differentiation, via mechanosensitive transcription factors (Swift, et al. (2013) Science 341:1240104; Meng, et al. (2018) Nature 560:655; Dupont, et al. (2011) Nature 474:179). While three-dimensional (3D) hydrogels for supporting cells have been described (see, e.g., U.S. Pat. Nos. 10,934,529 B2, 11,065,362, WO 1998/012228 A1, WO 2006/044342 A2, WO 2008/157318 A2, WO 2012/112982 A2, WO 2014/025312 A1, WO 2016/004068 A1, WO 2016/019391 A1, and WO 2017/036533 A1), studies have revealed the importance of matrix properties in affecting cellular functions at the tissue scale by interfacing a population of cells with a bulk material. The prospect of using the mechanical environment to program cell fate is attractive because it readily mimics the endogenous physiological situation, does not require additional chemical cocktails, and is readily scalable. Efforts to achieve physical control of cell fate via materials have generally involved engineering spatiotemporal mechanical control within hydrogels that interface with cells (Lenzini, et al. (2019) Front. Bioeng. Biotechnol. 7:260). For example, it was shown that spatial patterning of material stiffness affects stem cell fate (Yang, et al. (2016) Proc. Natl. Acad. Sci. USA 113:E4439). Additionally, dynamic control of material stiffness has been achieved in various systems (Khetan, et al. (2013) Nat. Mat. 12:458; Accardo, et al. (2015) Proc. Natl. Acad. Sci. USA 324:59; Guvendiren & Burdick (2012) Nat. Commun. 3:792), which have shown to influence cellular functions. In addition, studies have shown that cells are highly sensitive to variations in local 3D matrix properties (Doyle, et al. (2015) Nat. Commun. 6:8720; Han, et al. (2018) Proc. Natl. Acad. Sci. USA 115:4075; Bao, et al. (2017) Nat. Commun. 8:1962); however, precisely how the local matrix directly surrounding cells affects cell fate decisions has not been previously described. Conventional approaches to interface a cell population with a bulk gel by uncontrolled mixing leads to heterogeneity in the local amount of the gel presented to single cells, which makes cell-material interactions difficult to control and subsequently study. In addition, to tune cell-to-material volume ratios using a bulk material, it is often necessary to change cell population density, confounding the interpretation of whether observed biological effects are due to changes in cell-material or cell-cell interactions. Thus, alternative approaches are necessary to precisely control how much material is locally presented to each cell in a 3D space to study cell fate decisions driven by the local matrix environment.

### SUMMARY OF THE INVENTION

This invention is alginate-coated cells composed of a cross-linked alginate hydrogel layer encapsulating single mesenchymal stem cells or progenitor cells, wherein the alginate is conjugated to one or more cell adhesive ligands and the cross-linked alginate hydrogel layer has a thickness of less than about 10 microns, e.g., about 0.5 to about 5 microns. In some aspects, the alginate hydrogel encapsulating the cells has a softness of between about 0.1 to about 10 kPa, preferably about 2 kPa. In other aspects, the alginate is cross-linked with a divalent or trivalent cation and/or the alginate has a molecular weight of greater than about 250 kDa, e.g., about 250 kDa to about 500 kDa. In other aspects, the alginate hydrogel layer further includes one or more growth factors (e.g., Bmp-2, Bmp2-derived agonist peptides, or BMP receptor agonists), inflammatory factors (e.g., TNFα, TNFα-derived agonist peptides, or TNF receptor agonists), differentiation factors, or a combination thereof. A composition including the alginate-coated cells and a pharmaceutically acceptable carrier or aqueous medium is also provided, which optionally includes one or more ion channel modulators, one or more cell contractility modulators, or a combination thereof.

This invention also provides a method of treating a subject in need of treatment with mesenchymal stromal cells or progenitor cells by administering to the subject the alginate-coated cells of the invention. In accordance with this aspect, the alginate-coated cells may be differentiated prior to administration to the subject. In other aspects, the subject is in need of skin, bone, cartilage, lung, heart, kidney, or blood vessel tissue repair and/or has lung fibrosis, muscle fibrosis, fibrosis of connective tissues, kidney fibrosis, liver fibrosis, corneal fibrosis, radiation-induced fibrosis, chronic graft versus host disease (GVHD)-induced fibrosis, systemic sclerosis, or myocardial infarction. Example of modes for administering the alginate-coated cells include intratracheal instillation, intratracheal inhalation, intravenous delivery, intramuscular delivery, intraarterial delivery, topical delivery, renal artery injection, portal vein injection, intrabone delivery, intraarticular delivery, intralymphatic delivery, intrathymic delivery, intrarenal delivery, intracorneal delivery, intraportal delivery, intrahepatic delivery, or intracardiac injection.

This invention further provides a method of preparing a composition comprising a cross-linked alginate hydrogel layer encapsulating single mesenchymal stromal cells or progenitor cells. In accordance with this method, an aqueous phase comprising mesenchymal stromal cells or progenitor cells and a divalent or trivalent cation is contacted with an oil phase comprising alginate conjugated to one or more cell adhesive ligands so that a cross-linked alginate hydrogel layer encapsulating single mesenchymal stromal cells or progenitor cells is formed. Preferably, the cross-linked alginate hydrogel layer has a thickness of less than about 10 microns, more preferably about 0.5 to about 5 microns, and the hydrogel layer thickness can be modulated without changing gel viscoelasticity.

### BRIEF DESCRIPTION OF THE DRAWINGS

FIG. 1A-FIG. 1C show the isotropic volume expansion of single cells modulated by varied gel deposition predicts stem cell differentiation. FIG. 1A, Quantification of alkaline phosphatase (ALP) activity with varied gel deposition after 7-day culture of MSCs in the osteogenic medium. n=3 independent experiments for each gel deposition and n=4 for the bulk gel. FIG. 1B, Gene expression of alp (left) and runx2 (right) after 10-day culture in the mixed osteogenic and adipogenic medium. n=3 independent experiments. For FIG. 1A and FIG. 1B, mean±S.E.M; *, FIG. 1A p=0.0013, FIG. 1B (left) p=0.0034, (right) p=0.0076 via ordinary one-way ANOVA followed by Tukey's multiple comparisons test. FIG. 1C, ALP activity vs. cell volume. Scr: scrambled, Piezolᵢ: Piezol siRNA, TRPV4ᵢ: TRPV4 siRNA. For FIG. 1C, cell volume at 6 hours after encapsulation is shown. The data were fit to a power-law equation and shown as mean±S.E.M.

FIG. 2A-FIG. 2B show the effect of BMP2 on osteogenesis of gel-coated MSCs. Mouse MSCs were encapsulated in RGD-alginate gel coating (5 μm thickness) with or without embedding a recombinant BMP2 protein (100 ng/ml). Cocktail refers to a set of small molecules used to induce osteogenic differentiation of MSCs. After 7-day culture, the mRNA expression of osteogenesis markers, including alp (FIG. 2A) and runx2 (FIG. 2B), was evaluated by qPCR and normalized to the house keeping gene gapdh. *p<0.05 one-way ANOVA, Tukey post-hoc analysis. n=3.

FIG. 3A-FIG. 3C show mouse MSCs in gel coating inhibit aberrant tissue remodeling after fibrotic lung injury. FIG. 3A, Microelasticity of lung tissue sections measured by atomic force microscopy. n=56 indentations of random regions for no bleo and n=90 for other groups. Nine tissue sections from 3 mice were measured. *, p<10⁻¹⁵ via Kruskal-Wallis one-way ANOVA followed by Dunn's multiple comparisons test. FIG. 3B and FIG. 3C, Quantification of neutrophils and lymphocytes, respectively, in bronchoalveolar lavage fluid. n=3 mice for no bleo and n=6 mice for other groups. *, FIG. 3B p=3.8×10⁵, FIG. 3C p=2.6×10⁶. Data are pooled from 3 independent experiments and indicated as mean±SD. Unless stated otherwise, p-values were derived from one-way Welch ANOVA followed by Dunnett T3 multiple comparisons test.

FIG. 4A-FIG. 4C show continuous presentation of recombinant TNFα in gel coating enables mouse MSCs to accelerate the resolution of fibrotic phenotypes. FIG. 4A, Microelasticity of lung tissue sections measured by atomic force microscopy. n=87 indentations of random regions for no bleo, n=117 for bleo+veh, and n=90 for other groups. Nine tissue sections from 3 mice. *, p<10⁻¹⁵ via Kruskal-Wallis one-way ANOVA followed by Dunn's multiple comparisons test. Quantification of neutrophils (FIG. 4B) and lymphocytes (FIG. 4C) in bronchoalveolar lavage fluid. n=5 mice for no bleo, n=6 mice for bleo veh, and n=7 mice for other groups; *, FIG. 4B p=2.1×10⁻⁷, FIG. 4C p=5.0×10⁻¹⁰. Data are pooled from 3 for FIG. 4A and 5 independent experiments for FIG. 4B and FIG. 4C, and indicated as mean±SD. Unless stated otherwise, p-values were derived from one-way Welch ANOVA followed by Dunnett T3 multiple comparisons test.

FIG. 5A-FIG. 5C show the efficacy of gel-coated MSCs in an animal model of muscle fibrosis. FIG. 5A, Collagen fibers of right muscles by two-photon analysis. n=10 randomly picked regions from each mouse. Welch's t-test, P<0.05. FIG. 5B shows average grip strength of each leg, and FIG. 5C shows hanging time of D2.mdx mice. Analyses were done before and 2 weeks after treatment of mice with either PBS only or gel-coated MSCs on the right muscle (μMSC). L: left, R: right. For grip strength test, each data point represents an average of 5 measurements from an individual mouse. For hanging time test, each data point represents an average of 3 measurements from an individual mouse. n≥3 mice, ±SD (***: P<0.001, ****, P<0.0001 with two-way ANOVA followed by Tukey's multiple comparisons test).

### DETAILED DESCRIPTION OF THE INVENTION

There is a need for therapeutic cell delivery systems that control microenvironmental cues of cells thereby optimizing their efficacy. A droplet microfluidic technique has now been developed to deposit a defined amount of hydrogel matrix around single cells to provide predefined chemomechanical cues to the cells and beneficial therapeutic outcomes. In particular, mesenchymal stromal cells (MSCs), when coated with a cross-linked alginate hydrogel layer undergo isotropic volume expansion more rapidly in thinner gels that present a cell adhesive peptide. Mathematical modeling and experiments show that MSCs experience higher membrane tension as they expand in thinner gels. Furthermore, thinner hydrogel coatings (e.g., 5 micron or less in thickness) facilitate osteogenic differentiation of MSCs and soft hydrogel coatings (e.g., approximately 2 kPa) enable MSCs to maintain normal collagen levels, fiber density, and microelasticity in a mouse model of fibrotic lung injury. Accordingly, the present invention provides alginate-coated MSCs or progenitor cells and methods of using the same in cell-based therapies.

As used herein, “mesenchymal stromal cells” (also referred to as “mesenchymal stem cell” or “MSCs”) are multipotent cells that can differentiate into a variety of progenitor cell types including connective tissue, bone, cartilage, and cells in the circulatory and lymphatic systems. Mesenchymal stromal cells are found in the mesenchyme, the part of the embryonic mesoderm that consists of loosely packed, fusiform or stellate unspecialized cells. Mesenchymal stromal cells can be obtained by conventional methods and can be identified one or more of the following markers: CD29⁺, CD31⁻, CD34⁻, CD44⁺ CD45⁻, CD51⁺, CD73⁺, CD90/Thy-1⁺, CD105⁺, CD166⁺, Integrin α1⁺, PDGF Rα⁺, Nestin⁺, Sca-1⁺, SCF R/c-Kit⁺, STRO-1⁺, and/or VCAM-1⁺. In some aspects, the mesenchymal stromal cells are derived or obtained from bone marrow (BM), skeletal muscle, lung tissue, cord blood, adipose tissue (ASC) and the like. In certain aspects, the MSCs can be obtained from the same tissue in which the alginate-coated cells are intended to subsequently be used to treat. For example, bone marrow-derived MSCs can be isolated, coated with alginate, and used for bone repair; skeletal muscle-derived MSCs can be isolated, coated with alginate, and used for the treatment of muscle fibrosis; and lung-derived MSCs can be isolated, coated with alginate, and used for the treatment of lung fibrosis. In particular aspects, the mesenchymal stromal cells are derived or obtained from human bone marrow.

As is conventional in the art, a “progenitor cell” is an early descendant of a stem cell that can differentiate to form one or more kinds of cells, but cannot divide and reproduce indefinitely. Progenitor cells include, e.g., neural progenitor cells that give rise to neurons, astrocytes and oligodendrocytes; hematopoietic progenitor cells that give rise to blood cells; myeloid progenitor cells that give rise to red blood cells/erythrocytes, platelets, mast cells, osteoclasts, granulocytes, monocyte-macrophages, and dendritic cells; and lymphoid progenitor cells that give rise to T-cells, B-cells, NK-cells and dendritic cells.

The cells of this invention may be autologous or heterologous, e.g., allogeneic. “Autologous” refers to a transplanted biological substance taken from the same individual. “Allogeneic” refers to a transplanted biological substance taken from a different individual of the same species.

The cells used in the preparation of the alginate-coated cells of the invention can be isolated and optionally purified. As used herein the term “isolated” is meant to describe a cell of interest that is in an environment different from that in which the element naturally occurs. “Purified” as used herein refers to a cell removed from an environment in which it was produced and is at least 60% free, preferably 75% free, and most preferably 90% free from other components with which it is naturally associated or with which it was otherwise associated with during production.

Purification and/or identification of cells of interest can be achieved through any means known in the art, for example, immunologically. Histochemical staining, flow cytometry, fluorescence-activated cell sorting (FACS), western blot analysis, enzyme-linked immunosorbent assay (ELISA), and the like may be used. Flow immunocytochemistry may be used to detect cell-surface markers and immunohistochemistry (for example, of fixed cells) may be used for intracellular or cell-surface markers. Western blot analysis may be conducted on cellular extracts. Enzyme-linked immunosorbent assay may be used for cellular extracts or products secreted into the medium. Antibodies for the identification of stem cell markers may be obtained from commercial sources, for example from Biolegend (San Diego, CA).

In accordance with this invention, individual or single cells are coated with a biocompatible, biodegradable matrix composed of a cross-linked alginate hydrogel. In this respect, each individual cell is encapsulated by a single layer of cross-linked alginate hydrogel, i.e., a spherical core-shell microcapsule. This is distinct from a plurality of cells embedded in a layer of hydrogel matrix. The hydrogel of this invention is engineered to retain and release bioactive substances from cells in a spatially and temporally controlled manner. This controlled release not only eliminates systemic side effects and the need for multiple injections/infusions, but can be used to create a microenvironment that activates host cells at a hydrogel implant site and transplanted cells seeded onto/into a hydrogel.

“Biocompatible” generally refers to a material and any metabolites or degradation products thereof that are generally non-toxic to the recipient and do not cause any significant adverse effects to the subject. “Biodegradable” generally refers to a material that will degrade or erode by hydrolysis or enzymatic action under physiologic conditions to smaller units or chemical species that are capable of being metabolized, eliminated, or excreted by the subject. The degradation time is a function of polymer composition and morphology.

For the purposes of this invention, a “hydrogel” refers to a substance formed when an organic polymer (natural or synthetic) is cross-linked via covalent, ionic, or hydrogen bonds to create a three-dimensional open-lattice structure which entraps water molecules to form a gel. “Biocompatible hydrogel” refers to a polymer which forms a gel which is not toxic to living cells, and allows sufficient diffusion of oxygen and nutrients to the entrapped cells to maintain viability.

“Alginate” is a collective term used to refer to linear polysaccharides formed from (1-4)-linked β-D-mannuronic acid monomers (M units) and L-guluronic acid monomers (G units) in any M/G ratio and sequential distribution along the polymer chain, as well as salts and derivatives thereof. In certain aspects, an alginate of use in the preparation of the hydrogel of this invention has a molecular weight of greater than about 250 kDa (e.g., about 251 kDa, about 300 kDa, about 350 kDa, about 400 kDa, about 450 kDa or about 500 kDa). Preferably, an alginate of use in the preparation of the hydrogel of this invention has a molecular weight in the range of about 250 kDa to about 500 kDa.

In some aspects the alginate monomers are cross-linked via ionic bonds to form the hydrogel layer encapsulating the individual cells. Unlike many other gelation procedures, ionic cross-linking can occur at pH, temperature and salt conditions that maintain cell viability and/or protein activity. Ionic cross-linking of alginate can be carried out using in the presence of one or more divalent or trivalent cations. Suitable divalent or trivalent cations include, e.g., Ca²⁺, Mg²⁺, Sr²⁺, Ba²⁺, Be²⁺, Al³⁺ or a combination thereof. In a particular aspect, the divalent cation is Ca²⁺, Sr²⁺, Ba²⁺, or a combination thereof. In a preferred aspect, the alginate monomers are cross-linked with Ca²⁺.

Alternatively, the alginate monomers are cross-linked via covalent bonds. Carboxylate groups present on the monomer chains provide a region for relatively straight forward modification. For example, alginate may be modified by functionalizing carboxylate moieties with methacrylates such as 2-aminoethyl methacrylate hydrochloride, which allows for photo crosslinking (Somo, et al. (2018) Acta Biomater. 65:53; Chou, et al. (2009) Osteoarthritis Cartilage 17(10):1377-84; Samorezov, et al. (2015) Bioconjug. Chem. 26(7):1339-47). One or a combination of ionic and covalent cross-linking can be used to modify the mechanical stability, stiffness, and/or utility of the hydrogel.

According to this invention, single or individual cells are coated with the cross-linked alginate hydrogel layer having a thickness of up to 10 microns. However, in certain aspects, the thickness of the cross-linked alginate layer is less than about 10 microns, less than 9, less than 8, less than 7, less than 6, less than 5 microns, or in particular aspects less than about 3 microns. In certain aspects, the cross-linked alginate hydrogel coating an individual MSC or progenitor cell has a thickness of about 0.5 to about 10 microns, about 0.5 to about 5 microns, 0.5 to about 4 microns, 0.5 to about 3 microns, 0.5 to about 2 microns, 0.5 to about 1 microns, 1 to about 2 microns, 1 to about 3 microns, 1 to about 4 microns, 1 to about 5 microns, 2 to about 3 microns, 2 to about 4 microns, 2 to about 5 microns, 3 to about 4 microns, 3 to about 5 microns, 4 to about 5 microns, 1.5 to about 2 microns, 1.5 to about 3 microns, 1.5 to about 4 microns, 1.5 to about 5 microns, 2.5 to about 3 microns, 2.5 to about 4 microns, 2.5 to about 5 microns, 3.5 to about 4 microns, 3.5 to about 5 microns, 4.5 to about 5 microns, about 3 to about 10 microns, about 3 to about 9 microns, or about 3 to about 7 microns. In a particular aspect, the cross-linked alginate hydrogel layer coating an individual MSC or progenitor cell has a thickness of about 0.5 micron to about 5 microns, or about 0.5 micron to about 3 microns.

As discussed herein, MSCs undergo isotropic volume expansion more rapidly in thinner gels including a cell adhesive ligand, MSCs experience higher membrane tension as they expand in thinner gels, and thinner gels facilitate osteogenic differentiation of MSCs and facilitate the use of MSCs in ameliorating fibrotic tissue injury. Accordingly, the cross-linked alginate hydrogel layer encapsulating the single mesenchymal stem cells or progenitor cells is further characterized by low stiffness, e.g., have Young's modulus of about 0.1 to about 10 kPa. In some aspects, the hydrogel has a Young's modulus of 0.1 to about 10 kPa, 0.1 to about 9 kPa, 0.1 to about 8 kPa, 0.1 to about 7 kPa, 0.1 to about 6 kPa, 0.1 to about 5 kPa, 0.1 to about 4 kPa, 0.1 to about 3 kPa, 0.1 to about 2 kPa, 0.1 to about 1 kPa, 0.5 to about 1 kPa, 0.5 to about 2 kPa, 0.5 to about 3 kPa, 0.5 to about 4 kPa, or about 0.5 to about 5 kPa. In a particular aspects, the cross-linked alginate hydrogel encapsulating the single mesenchymal stem cells or progenitor cells has a Young's modulus of about 2 kPa.

In some aspect, the alginate hydrogel coating is characterized by a stress relaxation rate (τ_{1/2}) of 10 seconds or less, 9 seconds or less, 8 seconds or less, 7 seconds or less, 6 seconds or less, 5 seconds or less, e.g., 4 seconds, 3 seconds, 2 seconds, 1 second, 0.5 second, 0.1 second or less). In some aspects, the alginate hydrogel coating is characterized by a stress relaxation rate in the range of about 0.1 second to about 10 seconds. Preferably, the alginate hydrogel coating is characterized by a stress relaxation rate (τ_{1/2}) of about 4 seconds.

The diameter of the encapsulated cells is typically in the range of about 7 to about 30 microns, or more preferably about 15 to 20 microns. As shown herein, the volume or size of encapsulated single cells depends on both the cell adhesion molecule and hydrogel coating thickness. For example, when cells are in thinner hydrogels (i.e., a cross-linked alginate hydrogel layer having a thickness of about 5 microns) that are functionalized with a cell adhesive ligand, they expand by 50% more rapidly than when cells are in thicker hydrogels (i.e., a cross-linked alginate hydrogel layer having a thickness of about 15 microns).

Accordingly, in particular aspects the cross-linked alginate hydrogel is conjugated to one or more cell adhesive ligands. As used herein, a cell adhesion ligand is a molecule that facilitates attachment of the alginate to the cell surface. Cell adhesive ligands are often polysaccharides and/or short peptide sequences derived or obtained from fibronectin, vitronectin, laminin, collagen, elastin, or thrombospondin. Examples of suitable cell adhesion polysaccharides include hyaluronic acid or chondroitin. Examples of suitable cell adhesion peptides include RGD or RGD-containing peptides such as RGDS (SEQ ID NO:31), RGDSP (SEQ ID NO:32), RGDSPK (SEQ ID NO:33), RGDTP (SEQ ID NO:34), RGDSPASSKP (SEQ ID NO:35), or GGGGRGDSP (SEQ ID NO:1) derived from fibronectin; KQAGDV (SEQ ID NO:36), PHSRN (SEQ ID NO:37), YIGSR (SEQ ID NO:38) or RLVSYNGIIFFLK (SEQ ID NO:2) derived from laminin; DGEA (SEQ ID NO:39) derived from collagen; or VAPG (SEQ ID NO:41) derived from elastin. Particularly preferred as cell adhesion ligands attached to alginate chains are synthetic peptides containing arginine-glycine-aspartate (RGD) amino acid sequences found in various natural extracellular matrix molecules.

Covalent conjugation or coupling of cell adhesion ligands to alginate can be performed using synthetic techniques commonly known to those skilled in the art and exemplified herein. A particularly useful method is by forming an amide bond between the carboxylic acid group on the alginate chain and the amino group on the cell adhesion molecule. Other useful adhesion chemistries include those discussed by Hermanson ((1996) Bioconjugate Techniques, p. 152-183).

In some aspects the cross-linked alginate hydrogel layer may further include one or more active ingredients such as growth factors, inflammatory factors, and/or differentiation factors. Such active ingredients may be included during the preparation of the alginate hydrogel layer or after encapsulation of the cells in the cross-linked alginate hydrogel layer. In the latter case, the active ingredient can be provided to the encapsulated cells and diffuse through the hydrogel gel.

As used herein, a “growth factor” is a substance, which stimulates the growth of living cells. Examples of suitable growth factors that may be included or embedded in the cross-linked alginate hydrogel layer are Bmp1, Bmp2, Bmp3, Bmp4, Bmp5, Bmp6, Bmp7, Bmp8A, Bmp8B, Clec11a, Ostn, Chrdl1, Col1a1, Col1a2, Col5a1, Col5a2, Col5a3, Col6a1, Col6a2, Col6a3, Col13a1, Ecm1, Pkdcc, Fn1, Fstl3, Gdf2, Gdf3, Gdf10, Igsf10, Ifitm1, Kazald1, LTF, Lrrc17, Mgp, Lamb3, TGFB1, TGFB3, PDGF, VEGF, PTH, IGF1, FGF2, FGF9, BGLAP2, BGLAP3, PRG2, MEPE, and the like, as well as agonistic peptides of the same. Agonists of receptors of the above referenced growth factors can also be used. In a particular aspect, the growth factor is Bmp2, a Bmp2-derived agonist peptide, or a BMP receptor agonist.

For the purposes of this invention “inflammatory factors” are substances can stimulate or suppress an inflammatory response. Representative examples of inflammatory factors of use in this invention include interleukins (e.g., IL4, IL1, IL6, and IL13), interferon gamma (IFNγ), TNFα, GM-CSF, or a combination thereof. In some aspects of this invention, the inflammatory factor is a Toll-like receptor (TLR) ligand that induces inflammatory factors. Examples of suitable TLR ligands include, poly(A:U), poly(I:C), aminoalkyl glucosaminide 4-phosphates, lipopolysaccharide (LPS), or a combination thereof. In a particular aspect, the inflammatory factors include, TNFα, TNFα-derived agonist peptides, and/or TNF receptor agonists.

As used herein, “differentiation factors” are substances involved in promoting cell differentiation into various cell types, e.g., bone, fat, blood or muscle. By way of illustration, dexamethasone, ascorbic acid or its derivative ascorbic acid-2-phosphate, beta-glycerophosphate, and optionally heparin, retinoic acid, and/or 1,25(OH)₂D₃ has been shown to stimulate osteogenic differentiation of MSCs. Similarly, insulin, dexamethasone, and 3-Isobutyl-1-methylxanthine (IBMX) are routinely used to is routinely used to differentiate adipocytes in culture. 5-Azacytidine (5-aza), salvianolic acid B (SalB), and cardiomyocyte lysis medium (CLM) are of use inducing MSCs to acquire the phenotypical characteristics of cardiomyocytes.

This invention also provides a method for preparing the alginate-coated cells of this invention. In accordance with this method, an aqueous phase containing mesenchymal stromal cells or progenitor cells and a divalent or trivalent cation is contacted with an oil phase containing alginate conjugated to one or more cell adhesive ligands so that a cross-linked alginate hydrogel layer encapsulating single mesenchymal stromal cells or progenitor cells is formed. Ideally, the alginate has a molecular weight of at least about 240 kDa; the cation is a divalent cation such as Ca²⁺, Sr²⁺ or Ba²⁺; the cell adhesive ligand contains the sequence Arg-Gly-Asp; the hydrogel layer has a Young's modulus of about 2 kPa; the cross-linked alginate hydrogel layer has a thickness of less than 10 microns, or more preferably about 0.5 microns to about 5 microns; and/or the hydrogel layer thickness can be modulated without changing gel viscoelasticity. Advantageously, this method can retain high levels of cell viability without the need to perform additional cell sorting. Moreover, gel thickness and softness can be modulated for different applications. For example, for bone regeneration the gel coating layer thickness should not be greater than 10 microns if softness is 2 kPa, but the thickness can be greater if softness is greater than 10 kPa. For fibrosis treatment, the gel coating layer thickness should be less than 5 microns, and softness should be ˜2 kPa but not be greater than 10 kPa.

As demonstrated herein, the alginate-coated cells of this invention are of particular use in cell-based therapies. Accordingly, this invention also provides a method of treatment by administering to a subject in need of treatment with mesenchymal stromal cells or progenitor cells (e.g., by injection or transplantation) an effective amount of the alginate-coated cells of this invention. As used herein, “subject” means an individual. Thus, subjects include, for example, domesticated animals, such as cats and dogs, livestock (e.g., cattle, horses, pigs, sheep, and goats), laboratory animals (e.g., mice, rabbits, rats, and guinea pigs) mammals, non-human mammals, primates, non-human primates, rodents, birds, reptiles, amphibians, fish, and any other animal. The subject is preferably a mammal such as a primate or a human.

The alginate-coated cells can be administered by injection, for example, intravenously, intra-muscularly, intra-arterially, intra-bone, intratracheally, and the like. In certain embodiments, administration involves providing to a subject about 10², 10⁴, 10⁶, 10⁷, 10⁸, 10⁹, 10¹⁰, 10¹², or more cells. The number of cells administered may be chosen based on the route of administration and/or the severity of the condition for which the cells are administered.

In additional aspects, alginate-coated cells are administered in conjunction with a second therapeutic agent, e.g., an agent useful in treating the subject's disease or condition. The second therapeutic agent is different from the present alginate-coated cells and may include cells, antibodies, proteins and peptides, or small molecules. In certain aspects, the second therapeutic agent is selected from ion channel modulators, cell contractility modulators, or a combination thereof. The alginate-coated cells and second therapeutic agent can be administered simultaneously or sequentially. In addition, the alginate-coated cells and second therapeutic agent can be administered from a single composition or two separate compositions. The second therapeutic agent is administered in an amount to provide its desired effect. The effective dosage range for each second therapeutic agent is known in the art or may be determined by routine experimentation, and the second therapeutic agent is administered to an individual in need thereof within such established range.

Compositions containing the alginate-coated cells can be prepared by combining the cells with a pharmaceutically acceptable carrier or aqueous medium. The phrase “pharmaceutically acceptable” or “pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and the like. The use of such media and agents for pharmaceutically active substances is well known in the art. Except insofar as any conventional media or agent is incompatible with the cells of the present disclosure, its use in therapeutic compositions is contemplated. Pharmaceutical compositions can be determined by one skilled in the art depending upon, for example, the intended route of administration, delivery format and desired dosage. See, for example, Remington, J. P. & Allen, L. V. (2013) Remington: The Science and Practice of Pharmacy. London, Pharmaceutical Press.

The compositions of the invention can be incorporated in an injectable formulation. The formulation may also include the necessary physiologically acceptable carrier material, excipient, lubricant, buffer, surfactant, antibacterial, bulking agent (such as mannitol), antioxidants (ascorbic acid or sodium bisulfite) and the like.

Acceptable formulation materials preferably are nontoxic to recipients at the dosages and concentrations employed. The pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials may include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA; complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as poloxamers, PEG, sorbitan esters, polysorbates such as polysorbate 20 and polysorbate 80, Triton™, trimethamine, lecithin, cholesterol, or tyloxapal); stability enhancing agents (such as sucrose or sorbitol); tonicity enhancing agents (such as alkali metal halides, preferably sodium or potassium chloride, mannitol, or sorbitol); delivery vehicles; diluents; excipients and/or pharmaceutical adjuvants. See, for example, Remington, J. P. & Allen, L. V. (2013) Remington: The Science and Practice of Pharmacy. London, Pharmaceutical Press.

The primary vehicle or carrier in a pharmaceutical composition may be either aqueous or nonaqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. Pharmaceutical compositions can include Tris buffer of about pH 7.0-8.5, or acetate buffer of about pH 4.0-5.5, which may further include sorbitol or a suitable substitute therefore. Pharmaceutical compositions of the invention may be prepared for storage by mixing the selected composition having the desired degree of purity with optional formulation agents (Remington, J. P. & Allen, L. V. (2013) Remington: The Science and Practice of Pharmacy. London, Pharmaceutical Press.) in the form of a lyophilized cake or an aqueous solution.

The cells or composition can be provided by sustained release systems, by encapsulation or by implantation devices. The compositions may be administered by bolus injection or continuously by infusion, or by implantation device. The composition also can be administered locally via implantation of a membrane, sponge or another appropriate material onto which the cell or cells have been absorbed or encapsulated. Where an implantation device is used, the device may be implanted into any suitable tissue or organ. The injections may be given as a one-time treatment, repeated (daily, weekly, monthly, annually etc.) to achieve the desired therapeutic effect.

The compositions of the invention can be delivered parenterally. When parenteral administration is contemplated, the therapeutic compositions for use in this invention may be in the form of a pyrogen-free, parenterally acceptable aqueous solution. A particularly suitable vehicle for parenteral injection is sterile distilled water. Preparation can involve the formulation with an agent, such as injectable microspheres, bio-erodible particles, polymeric compounds (such as polylactic acid or polyglycolic acid), beads or liposomes, that may provide controlled or sustained release of the cell or cells, which may then be delivered via a depot injection. Formulation with hyaluronic acid has the effect of promoting sustained duration in the circulation. Implantable drug delivery devices may be used to introduce the desired composition.

These compositions may also contain adjuvants such as preservative, wetting agents, emulsifying agents and dispersing agents. Prevention of the action of microorganisms can be ensured by the inclusion of various antibacterial and antifungal agents, for example, paraben, chlorobutanol, phenol sorbic acid and the like. It may also be desirable to include isotonic agents such as sugars, sodium chloride and the like.

The active compositions of the present disclosure may include classic pharmaceutical preparations. Administration of these compositions according to the present disclosure will be via any common route so long as the target tissue is available via that route. Such routes include oral, nasal, buccal, rectal, vaginal or topical route. Ideally, administration may be by intratracheal instillation, intratracheal inhalation, intravenous delivery, intramuscular delivery, intraarterial delivery, topical delivery, renal artery injection, portal vein injection, intrabone delivery, intraarticular delivery, intralymphatic delivery, intrathymic delivery, intrarenal delivery, intracorneal delivery, intraportal delivery, intrahepatic delivery, or intracardiac injection of the alginate-coated cells. Such compositions would normally be administered as pharmaceutically acceptable compositions.

As used herein, the term “amount effective,” “effective amount” or a “therapeutically effective amount” refers to an amount of the cells or composition of the invention sufficient to achieve the desired result. The amount of the cells or composition which constitutes an “effective amount” or “therapeutically effective amount” may vary depending on the severity of the disease, the condition, weight, or age of the patient to be treated, the frequency of dosing, or the route of administration, but can be determined routinely by one of ordinary skill in the art. A clinician may titer the dosage or route of administration to obtain the optimal therapeutic effect.

Subjects in need of treatment in accordance with this invention include those with a disease or condition who would benefit from the administration of mesenchymal stromal cells or progenitor cells. In some aspects, the alginate-coated MSCs or progenitor cells are differentiated prior to being administered to the subject. In some aspects, the disease or disorder is caused by or involves the malfunction of hormone-or protein-secreting cells in a subject. In accordance with such aspects, hormone- or protein-secreting cells are encapsulated and administered to the subject. For example, encapsulated cells can be administered to a subject with diabetes. In other aspects, the cells are used to repair tissue in a subject. In accordance with such aspects, the cells form structural tissues, such as skin, bone, cartilage, muscle, lung, heart, kidney, or blood vessel tissue. In certain aspects, the alginate-coated cells are administered to a subject to promote osteogenesis in the subject. In other aspects, the alginate-coated cells are administered to a subject to treat fibrosis in the subject, e.g., lung fibrosis, muscle fibrosis, fibrosis of connective tissues, kidney fibrosis, liver fibrosis, corneal fibrosis, radiation-induced fibrosis, chronic graft versus host disease (GVHD)-induced fibrosis, systemic sclerosis, or myocardial infarction. In addition, the alginate-coated cells of this invention are of use in the treatment of systemic sclerosis and fibrosis from chronic and acute graft-versus-host disease.

Although not precluded, treating a disease or condition does not require that the disease, condition, or symptoms associated therewith be completely eliminated, including the treatment of acute or chronic signs, symptoms and/or malfunctions. “Treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. “Treatment” therefore also includes relapse prophylaxis or phase prophylaxis. The term “treat” and synonyms contemplate administering a therapeutically effective amount of the alginate-coated cells of the invention to an individual in need of such treatment. A treatment can be orientated symptomatically, for example, to suppress symptoms. Treatment can be carried out over a short period, be oriented over a medium term, or can be a long-term treatment, for example within the context of a maintenance therapy.

The following non-limiting examples are provided to further illustrate the present invention.

### EXAMPLE 1: ALGINATE-COATED MESENCHYMAL STEM CELLS FOR PROMOTING OSTEOGENESIS

Cell Culture. Clonally derived D1 mouse MSCs were purchased from American Type Cell Culture. D1 MSCs were cultured in complete medium composed of high-glucose Dulbecco's Modified Eagle Medium (DMEM; Thermo Fisher Scientific) supplemented with 10% fetal bovine serum (FBS; Atlanta Biologicals), 1% penicillin-streptomycin (P/S), 1% GlutaMAX (Thermo Fisher Scientific). Cells were passaged when they reached ˜80% confluence by detaching with trypsin-EDTA (Thermo Fisher Scientific). D1 MSCs with passage number less than 13 were used in the study.

Alginate Preparation. Sodium alginate with ˜240 kDa molecular weight (LF200) was purchased from FMC Biopolymer. To enable cell adhesion to alginate, an integrin-binding peptide consisting of Arg-Gly-Asp (GGGGRGDSP (SEQ ID NO:1); Peptide 2.0) or a CD44-binding peptide A5G27 (RLVSYNGIIFFLK (SEQ ID NO:2; Peptide 2.0) was covalently conjugated to alginate by 1-ethyl-dimethylaminopropyl (EDC) and N-hydroxy sulfosuccinimide (NHS) (Sigma) chemistry with a degree of substitution of ˜20 according to known methods (Rowley, et al. (1999) Biomaterials 20:45). After conjugation, alginate was dialyzed against decreasing concentrations of NaCl, charcoal-treated, filter-sterilized, and lyophilized. Lyophilized alginate was stored in −20° C. and dissolved in DMEM within 1 week prior to experiments. To visualize alginate gels, a small amount (final w/v=0.05%) of 10/60 alginate (˜120 kDa; FMC Biopolymer) coupled with Lissamine™ Rhodamine B Ethylenediamine (Thermo Fisher Scientific) was added prior to gel formation.

Microfluidic Device Fabrication. Microfluidic devices were fabricated using soft lithography (Qin, et al. (2010) Nat. Protoc. 5:491). To develop a photoresist, SU-8 3025 (MicroChem) was deposited onto a silica wafer to a defined height and cured by UV light exposure through a transparency mask (CAD/Art Services) for patterning. Polydimethylsiloxane (PDMS; Dow Corning) was then mixed with crosslinker at ratio 10:1, degassed, poured, and cured for at least 3 hours at 65° C. The cured PDMS was peeled off the wafer and bonded to a glass slide by oxygen-plasma treatment of both surfaces. Microfluidic channels were then treated with fluoroalkylsilanes sold under the tradename AQUAPEL® (PPG Industries) and dried. Polyethylene tubing (inner diameter: 0.38 mm; outer diameter 1.09 mm) and 27G×½ needles were used to connect microfluidic channels to syringes (Becton Dickinson). Aqueous and oil flow rates in syringes were controlled by syringe pumps (Harvard Apparatus).

Tuning Alginate Gel Deposition Around Single Cells. CaCO₃ nanoparticles (CalEssence; 900 nm diameter) were resuspended in complete medium and dispersed by sonication with Vibra Cell Sonicator at 75% amplitude for 1 minute. The nanoparticles were then centrifuged at 50 g for 5 minutes to discard larger aggregates, followed by 1000 g for 5 minutes for collection. Purified CaCO₃nanoparticles were resuspended with serum-free DMEM medium; the concentration of CaCO₃ was increased from 4.8 to 27.0 mg/ml with thicker alginate gel deposition. Cells were then incubated with CaCO₃ by rotation at room temperature for 1 hour. Excess CaCO₃ nanoparticles were then washed out by centrifugation. The aqueous phase was prepared by resuspending CaCO₃-coated cells in buffer composed of DMEM with 50 mM HEPES, 10% FBS, 1% P/S at pH 7.4, and mixing cells with 1% w/v alginate solution. The oil phase was composed of fluorinated oil (3M) with 1% perfluoropolyether (PFPE, Miller Stephenson) as a surfactant and 0.03% acetic acid as an initiator of Ca²⁺ release from CaCO₃. The aqueous and oil phases were injected into the microfluidic device. For thicker alginate gel deposition, channel dimensions of the microfluidic device and flow rates were increased as noted in Table 1. Emulsion was collected every 20 minutes followed by 40 minutes rotation at room temperature. Emulsion was then broken by the addition of 10% 1H, 1H, 2H, 2H-perfluoroctanol (Alfa Aesar). Gel-coated cells were washed twice with serum-free DMEM. Roughly 12000 gel-coated cells were embedded in 50 μl of 1.25 mg/ml collagen-I matrix (Rat tail, Gibco/Thermo Fisher Scientific) on a 48-well glass bottom plate (MatTek Corporation), followed by culture at 37° C. in complete DMEM.

TABLE 1

| | Gel Volume (μm³) | | | |
|---|---|---|---|---|
| Conditions | ~5 × 10³ | ~1 × 10⁴ | ~2.5 × 10⁴ | ~5.5 × 10⁴ |
| Device Height (μm) | 15 | 15 | 30 | 30 |
| Device Width (μm) | 10 | 20 | 30 | 50 |
| Aqueous flow rate | 1 | 1 | 1 | 3 |
| (μl/minute) | | | | |
| Oil flow rate | 3 | 3 | 3 | 6 |
| (μl/minute) | | | | |
| CaCO₃ (mg/ml) | 4.8 | 9.6 | 9.6 | 27 |

Cell Encapsulation in Bulk Alginate Hydrogels. Cells were resuspended in 1% w/v LF200 alginate in DMEM, and rapidly mixed with calcium sulfate by syringe. A final concentration of 10 mM calcium sulfate was used to form the bulk hydrogel with E˜2 kPa. The mixed solution was deposited between two glass plates with a 1 mm void thickness. After 1.5 hours, hydrogels were punched into discs and cultured in a 96-well glass bottom plate (MekTak) in complete medium.

Mechanical Analysis of Gel Deposition Around Cells. Gel-coated cells were immobilized on a glass slide pre-coated with 0.1 mg/ml of poly-L-lysine for 2 hours. The slide was then placed in an MFP-3D system (Asylum Research) to perform atomic force microscopy (AFM) with a silicon nitride cantilever with an 18° pyramid tip (MLCT, Bruker). A spring constant of the cantilever was determined from thermal fluctuations at room temperature (20˜40 mN/m) before each analysis. A fluorescent microscope was used to bring the cantilever to the gel surface. Indentation was then performed under contact mode with force distance 500 nm and 1 μm/s velocity until the trigger cantilever deflection voltage (0.5 V) was reached, followed by retraction. To calculate Young's modulus (E), force-indentation curves were fitted to the Hertzian model with a pyramid indenter (Darling, et al. (2006) Osteoarthritis Cartilage 14:571; de Sousa, et al. (2020) Sci. Rep. 10:4749) and Poisson's ratio (v)=0.5.

Stress relaxation times of gels with varied deposition were measured by AFM according to established methods (Darling, et al. (2006) Osteoarthritis Cartilage 14:571; de Sousa, et al. (2020) Sci. Rep. 10:4749). The cantilever was brought toward the gel surface at velocity 1 μm/s. Once the trigger cantilever deflection voltage (0.5V) was reached, z-height was increased to 100 nm, followed by a 9 second dwell time, and then the cantilever was retracted. The deflection of the tip during the dwell period was recorded under a constant load with the sampling rate=120 Hz. Values for tip deflection over the dwell period were converted to force using the spring constant measured during AFM calibration. For all time t, force (F) was converted to stress (S) using the equation for pyramidal tip geometry (de Sousa, et al. (2020) Sci. Rep. 10:4749):

$$S ( t ) = \frac{F ( t ) \sqrt{2} ( 1 - v )}{{γ}_{0}^{2} tan ( α )}$$

where v is Poisson's ratio (assumed to be 0.5 for hydrogels), γ₀ is the constant strain over the dwell period, and α is the pyramidal face angle. Stress curves over the dwell period were then fit to the equation:

$$S ( t ) = {E}_{R} ( 1 + \frac{{τ}_{σ} - {τ}_{ε}}{{τ}_{ε}} {e}^{- \frac{t}{{τ}_{ε}}} )$$

as described (Darling, et al. (2006) Osteoarthritis Cartilage 14:571), where E_{R} is the relaxed modulus, τ_{σ} is the time of relaxation of deformation under constant load, and τ_{ε} is the time of relaxation of load under constant deformation. E_{R} can be calculated from Young's modulus from force-indentation curves, since Young's modulus=1.5E_{R} (Darling, et al. (2006) Osteoarthritis Cartilage 14:571).

Confocal Imaging and Image Analysis. Cells in gels containing alginate-rhodamine were incubated with 1 μM of Hoechst 33342 and 2 μM of calcein AM (both from Thermo Fisher Scientific) for 1 hour to stain nucleus and cytoplasm, respectively. Samples were then washed with HBSS and maintained in Fluorobrite DMEM (Thermo Fisher Scientific) at 37° C. 5% CO₂ during confocal imaging in the Zeiss LSM 770 system with a motorized stage and the 20×/0.8 M27 Plan-Apochromat objective. To analyze cell volume, z-stacks were captured with 60-90 μm total depth with each image at 0.77 μm for 75-115 images per z-stack. The stacks were then analyzed in IMARIS® (Bitplane, version 7.7.2). 3D reconstruction of each stack was performed by the built-in algorithm. Voxels were generated for red (alginate-rhodamine), green (calcein), and blue (Hoechst) signals after automatic thresholding. Thresholding values varied less than 10% across all the images from different experiments. A gel-coated cell was considered an outlier and hence excluded from the analysis if it met one of the following criteria: 1) blue voxels extending beyond the boundary of green voxels; 2) green voxels extending beyond the boundary of red voxels; 3) red voxels not containing green or blue voxels inside; or 4) green and blue voxels not within red voxels. The total voxels above the threshold were then calculated to quantify gel, cytoplasmic and nuclear volumes of each gel-coated cell. Sphericity of gel, cell, and nucleus was analyzed from the same set of voxels and defined as (π^{1/3}(6·V)^{2/3})/A, where V is volume and A is surface area.

Chemical Inhibitors. The following chemical inhibitors were purchased from Cayman Chemical: GSK1016790A and HC-067047. GsMTx-4 was purchased from Alomone Labs.

Measurement of Extracellular Ca²⁺ Concentration. A calcium assay kit (Cayman Chemical) was used to evaluate Ca²⁺ concentration in the culture medium according to the manufacturer's protocol, based on a colorimetric reaction between o-Cresolphthalein and calcium. The absorbance of the purple color was measured at 575 nm.

Finite Element Analysis to Model Gel Stress. In the case of large deformation with rubber-like elasticity, a nonlinear finite element method was applied to solve the boundary value problem using the commercial finite element package Abaqus. The axisymmetric formulation was used to solve the 3D problem. Approximately 2000 quadrilateral axisymmetric elements were used to reach convergence. The displacement boundary condition (u=u₀) was applied at the inner boundary, and the stress-free boundary condition (σᵣᵣ(r=r₁+d_{gel})=0) was applied at the outer boundary. The incompressible neo-Hookean material was used to consider the rubber-like elasticity of the gel. The strain energy potential of neo-Hookean material is given as

$$U = \frac{G}{2} ( {I}_{1} - 3 ) ,$$

where I₁=λ₁²+λ₂²+λ₃² is the first invariant of deformation, λ₁, λ₂, λ₃ are the principal stretches, and G is the shear modulus. Stress fields were then visualized using the ABAQUS CAE postprocessing interface.

Measurement of Membrane Tension by Fluorescent Lifetime Imaging Microscopy (FLIM). Cells in gels were incubated with 1 μM of FLIPPER-TR® lipid membrane tension probe (Cytoskeleton, Inc.) (Colom, et al. (2018) Nat. Chem. 10:1118) in complete medium for 30 minutes. FLIM was performed in the Ultima Multiphoton Microscope System equipped with a Becker and Hickl time correlated single-photon counting module (Bruker). The probe was excited at 920 nm by the Chameleon Ultra II Two-Photon laser operating at 80 MHz. The emission signal was collected through a bandpass 595/50 nm filter for 1 minute. Signal decay time (τ) values were extracted by fitting the average photon count versus time graph to a two-phase exponential decay fit in the data analysis software SPCimage (Becker & Hickl GmbH); τ values correspond to the first component of the lifetime (τ₁) in the curve fit, since the second component accounts for a minority of the signal.

Retrieval of Cells from Gels. Cells in gels were retrieved by digesting with 2.5 mg/ml collagenase P (Sigma), 4 mg/ml alginate lyase (Sigma), and 0.125% trypsin-EDTA (Thermo Fisher Scientific) at 37° C. for 30 minutes. Samples were then centrifuged at 3000 rpm for 5 minutes and washed twice with HBSS, followed by downstream analyses.

Cell Viability Analysis by Flow Cytometry. Cells retrieved from gels were added to the stain buffer composed of HBSS with 2 μM of calcein AM, Biotium) and 2 μM ethidium Homodimer-1 (Thermo Fisher Scientific) for 30 minutes. Samples were then analyzed by flow cytometry using BD LSRFORTESSA® (Becton Dickinson). An event threshold of 5000 in forward scatter was used to exclude debris. Percent cell viability was calculated by dividing the number of calcein⁺ ethidiu⁻ events by the total event number. In some cases, APC beads (Calibrite™; Becton Dickinson) with a known number were added in each sample to calculate an absolute number of viable and dead cells.

Diffusion Assay. To characterize the diffusion kinetics with varied gel deposition, small (˜25 μm in diameter) or large (˜45 μm in diameter) gels without cells were generated by mixing alginate with 4.8 mg/ml CaCO₃ and running through the droplet microfluidic device by using the same parameters as single cell encapsulation, followed by confirmation of Young's modulus by AFM (E˜2 kPa). Small, large, and bulk alginate gels were then incubated with fluorescein isothiocyanate-dextran (FITC-dextran) with average molecular weight ˜20 kDa (Sigma). The media were collected, and gels were digested after incubation by using the cell retrieval protocol at different time points: 30, 60, 120 and 1440 minutes. FITC-dextran in media and digested gels were then measured in a black 96-well plate at excitation/emission=490/520 nm by using PHERAstar® (Version 5.41).

MSC Differentiation and Alkaline Phosphatase Activity Assay. To evaluate the differentiation potential of MSCs in gels 1 day after encapsulation, they were cultured in medium supplemented with either an osteogenic chemical cocktail alone or both osteogenic and adipogenic cocktails for 7 or 10 days, respectively. All reagents for MSC differentiation were purchased from R&D Systems. One half of each sample was used to quantify an absolute number of viable cells by flow cytometry as described above, while the other half was used to evaluate alkaline phosphatase (ALP) activity. To quantify ALP activity, samples were lysed with 100 μl passive buffer (Promega) for at least 10 min at 4° C. Each lysate was then added to a black 96-well plate pre-loaded with 100 μl 4-Methylbelliferyl phosphate (4-MTP) substrate (Sigma). Signals were acquired with excitation at 360 nm and emission at 450 nm using a plate reader. Recombinant mouse ALP protein (Novus Biologicals) was used to generate a standard curve for calibration. ALP activity of each sample was then normalized to the number of viable cells.

Gene Expression Analysis. Cells were lysed with 1 mL of acid-guanidinium-phenol based reagent sold under the tradename TRIZOL® (Thermo Fisher Scientific) for 10 minutes. Samples in TRIZOL® reagent were stored at −80° C. if not processed immediately up to 1 week. Chloroform (200 μl) was added per 1 ml TRIZOL® reagent for phase separation. Samples were centrifuged for 15 minutes at 12,500 rpm, 4° C. The top layer containing RNA was collected into a new tube, and then precipitated with 250 μl isopropanol, and 250 μl 0.8 M sodium citrate combined with 1.2 M sodium chloride for at least 15 minutes at 4° C. Samples were then centrifuged at 12,500 rpm for 15 minutes at 4° C. The supernatant was removed, and the precipitated RNA was washed with 75% ethanol, followed by centrifugation for 5 minutes at 7,500 rpm, 4° C. After removing ethanol, purified RNA was resuspended in 15 μl of RNase-free water (Thermo Fisher Scientific). NanoDrop spectrophotometer (Thermo Fisher Scientific) was used to quantify RNA concentration and quality. cDNA was obtained by reverse transcription using SUPERSCRIPT®-III reverse transcriptase (Thermo Fisher Scientific). For each sample, 50 ng cDNA was added to each well in triplicate, followed by the Power SYBR Green PCR Master Mix (Applied Biosystems). Quantitative PCR was performed in the ViiA7 qPCR system (Thermo Fisher Scientific). Relative gene expression was calculated using the AACt method by comparing each cycle threshold (Ct) value to the reference gene (gapdh). Primer sequences are listed in Table 2.

TABLE 2

| Gene | | | SEQ ID |
|---|---|---|---|
| (Accession No.) | Primer | Sequence | NO: |
| Gapdh | Forward | CTTTGTCAAGCTCATTTCCTGG | 3 |
| (NM_001289726.1) | Reverse | TCTTGCTCAGTGTCCTTGC | 4 |
| Alp | Forward | CTCCAAAAGCTCAACACCAATG | 5 |
| (NM_001287172.1) | Reverse | ATTTGTCCATCTCCAGCCG | 6 |
| runx2 | Forward | GCTATTAAAGTGACAGTGGACGG | 7 |
| (NM_001146038.2) | Reverse | GGCGATCAGAGAACAAACTAGG | 8 |
| pparg1 | Forward | TGTTATGGGTGAAACTCTGGG | 9 |
| (NM_001127330.2) | Reverse | AGAGCTGATTCCGAAGTTGG | 10 |

RNA Interference. Small interfering RNAs (siRNAs) were purchased from Thermo Fisher Scientific as follows: piezol (Assay ID: 502463), trpv4 (Assay ID: 182204), and scrambled (Silencer negative control no. 1 siRNA). siRNA with concentration 4 nM was mixed with transfection reagent sold under the tradename LIPOFECTAMINE® RNAiMAX® (Thermo Fisher Scientific) for 15 minutes in culture medium sold under the tradename Opti-MEM® (Thermo Fisher Scientific). The mixture was then applied to cells and cultured for 3 days. Quantitative PCR was used to confirm the knockdown efficacy of each target gene compared to the scrambled control.

Statistical Analysis. Statistical hypothesis tests were performed in GraphPad Prism. Where standard deviations did not vary between test groups, one-variable analysis was performed using ordinary one-way ANOVA followed by Tukey multiple comparison testing. Where standard deviations were variable, one-variable analysis was performed using one-way Welch's ANOVA followed by Dunnett T3 multiple comparison testing. For two-factor analysis, such as cell volume as a function of time, repeated measures two-way ANOVA with Sidak's multiple comparison testing were used. A p-value less than 0.05 established statistical significance.

Investigation of the Effect of Varied Gel Deposition on in vivo MSC-Based Bone Regeneration. To test whether increased osteogenesis of MSCs in thinner gels corresponds to enhanced regenerative outcomes in vivo, engineered MSCs are delivered to a mouse model of mechanical marrow ablation. This model allows for the evaluation of the direct effect of engineered MSCs on bone formation without any cartilage precursor, i.e., intramembranous bone formation.

C57BL6/J mice between 8˜10 weeks old are used. After drilling a hole in the patellar groove of both femurs and flushing the marrow cavity with 60 μl saline solution, the ablated space is allowed to fill with blood and clot, followed by delivery of samples into the cavity (in 10 μl saline solution). A total of 10 groups are examined (Table 3). Primary bone marrow MSCs are derived from C57BL6/J mice and coated in thin (5 μm) or thick (15 μm) alginate-RGD gel (240 kDa, 1% w/v, [RGD]=750 μM, E=2 kPa). Since hydrogel degradation is desirable for donor cell integration in vivo, MSCs encapsulated in partially oxidized (5%) alginate-RGD gels, which are known to be degraded within 9 days by hydrolysis, are also tested. Degradation is confirmed in vitro by imaging of fluorescent alginate microgels and tuned when necessary. Using the droplet microfluidic approach described herein, acellular alginate-RGD microgels are generated as negative controls that correspond to the total diameter of thin and thick gel-coated MSCs, 28 and 48 μm, respectively, given that MSCs show ˜18 μm in diameter after 50% volume expansion.

TABLE 3

| Group # | Group Description |
|---|---|
| 1 | MSC in regular thin (5 μm) gel |
| 2 | MSC in regular thick (15 μm) gel |
| 3 | MSC in oxidized thin gel |
| 4 | MSC in oxidized thick gel |
| 5 | MSC only |
| 6 | 28 μm acellular regular microgel |
| 7 | 48 μm acellular regular microgel |
| 8 | 28 μm acellular oxidized microgel |
| 9 | 48 μm acellular oxidized microgel |
| 10 | Untreated ablation |

Biodistribution Kinetics of Donor MSCs and Gel Coating. To assess biodistribution of MSCs in live mice, MSCs are transduced with a retroviral vector to express both firefly luciferase and mCherry (Addgene; luc⁺FmC⁺ MSCs), followed by sorting and expanding the transduced cells. Luc⁺mC⁺ MSCs are then encapsulated in alginate-RGD gel coating (Group #1-4, Table 3) conjugated with fluorescent dye sold under the tradename ALEXA FLUOR® 750 by using carbodiimide chemistry. The luc⁺mC⁺ MSC only group (Group #5) is also tested as a control. After delivering 5×10⁵ (˜5% of total cells in mouse femur) engineered luc⁺mC⁺ MSCs in one of the ablated femurs per 20 g body weight, both donor MSC and gel signals are acquired once every other day by luminescence and fluorescence (Ex: 745 nm, Em: 780 nm), respectively, with an IVIS system. The other ablated femur is also tracked to confirm persistent localization of donor MSCs in the injected side. At day 21, the mice are sacrificed to characterize engrafted mCherry⁺ donor cells in each femur by using intracellular flow cytometry with antibodies against CD146 (MSC) and osteocalcin (osteoblast).

Bone Regeneration. To quantify the speed of bone regeneration in vivo, all of the groups in Table 3 are tested by injecting 5×10⁵ gel-coated MSCs or microgels per 20 g mouse in one of the ablated femurs. Femurs are harvested at two time points (day 7 and 21). To quantify bone formation, micro-CT is performed using a Scanco Model μCT50 with 6 μm isotropic voxels (Scanco Medical AG; Rush University MicroCT and Histology Core) at 55 kVp tube voltage, 500 ms integration time, and 200 μA tube current. 3D reconstructions are generated with the manufacturer's software. Bone volume per total volume (BV/TV) in the medullary space from 30-60% of the total bone length is quantified, since this is the site of intramembranous bone regeneration after marrow ablation. Uninjected and injected femurs for each mouse are compared to assess whether accelerated bone regeneration is local to the injected side or global. Select samples are formalin-fixed, decalcified in 19% EDTA, and paraffin-embedded for histological analyses to evaluate tissue morphology by hematoxylin/eosin and Masson's trichrome staining.

Results. A droplet-based microfluidic approach was developed to decouple the amount of hydrogel deposition around single cells from material composition and elasticity. Because CaCO₃ nanoparticles are coated on cell surface, gelation of alginate only occurs in droplets that contain cells (Mao, et al. (2017) Nat. Mater. 16:236). By tuning the flow rates of aqueous and oil phases, the channel size of the microfluidic device, cell density in aqueous alginate solution, CaCO₃ and acetic acid concentrations, single murine mesenchymal stem cells (MSCs) were encapsulated with varied gel deposition around cells (gel thickness: 2-15 μm; gel volume: 2000˜45000 μm³; total droplet size: 20˜45 μm). The polymer concentration was kept at 1% w/v of ˜240 kDa alginate, and Young's modulus (E) was maintained at ˜2 kPa (Buxboim, et al. (2017) Mol. Biol. Cell 28:3333; Liu, et al. (2010) J. Cell Biol. 190:693). Furthermore, stress relaxation times were not altered by gel deposition, and in comparison to ‘rubber’-like materials previously described (U.S. Pat. No. 11,065,362 B2), the instant gel coating advantageously has a stress relaxation time of about 4 seconds. [Ca²⁺] in the medium remained physiological (˜2 mM) across the different experimental groups. Cross-linking of the polymer occurred simultaneously with droplet formation, which helped maintain cell viability after encapsulation in the gel with varied deposition. The estimated swelling ratio (Qᵥ) of gels remained the same (˜1.5) regardless of their size, which was expected for a constant w/v % and polymer cross-linking (Lee & Mooney (2012) Prog. Poly. Sci. 37:106). Thus, this approach enables tunable local 3D gel deposition around single cells in a deterministic manner.

Using the methodology described herein, volume regulation of single cells as a function of local 3D matrix deposition was assessed. MSCs were chosen as a model cell because they have been extensively investigated to understand cell-matrix interactions (Engler, et al. (2006) Cell 126:677; Huebsch, et al. (2010) Nat. Mat. 9:518; Khetan, et al. (2013) Nat. Mat. 12:458; Chaudhuri, et al. (Nat. Mat. 15:326; Fu, et al. (2010) Nat. Meth. 7:733; Guo, et al. (2017) Proc. Natl. Acad. Sci. USA 114:E8618; Lee, et al. (2019) Nat. Comm. 10:529; Swift, et al. (2011) Nature 474:179; Mao, et al. (2017) Nat. Mater. 16:236; Buxboim, et al. (2017) Mol. Biol. Cell 28:3333). Clonally derived murine D1 MSCs were used, since they provide less cell-to-cell heterogeneity compared to primary cells (Huebsch, et al. (2010) Nat. Mat. 9:518; Chaudhuri, et al. (Nat. Mat. 15:326; Guo, et al. (2017) Proc. Natl. Acad. Sci. USA 114:E8618; Lee, et al. (2019) Nat. Comm. 10:529). Single MSCs were encapsulated within the alginate gel with varied deposition: 9.6 (‘thin’), 20.0 (‘medium’) or 57.0 (‘thick’)×10³ μm³ in gel volume. The alginate gel-coated MSCs were subsequently embedded in collagen-I gel at a sparse density (5,000 cells in 20 μl) followed by confocal imaging analysis of live cells to evaluate their volume change over time. Gel-coated MSCs were compared with MSCs encapsulated in a bulk alginate gel at the same cell density, composition, and E. Molecular weight ˜240 kDa alginate was selected since single cells encapsulated in this gel formulation do not proliferate but remain viable in culture (Mao, et al. (2017) Nat. Mater. 16:236). Without any adhesion ligand, the volume of cytoplasm and nucleus was ˜1000 μm³ each, regardless of varied gel. Uncoated MSCs embedded in collagen-I within ˜2 hours also showed similar volume. Thus, 1000 μm³ was deemed to be the baseline volume (V₀) of cytoplasm and nucleus.

MSCs were then encapsulated in the alginate gel conjugated to the Arg-Gly-Asp (RGD) ligand, which binds to α₅β₁ and αᵥβ₃ integrins (alginate-RGD). The volume of gel deposition remained unchanged over 3 days in culture. In contrast to the adhesion ligand-free gel, MSCs in the thin alginate-RGD gel rapidly (t_{1/2}=1˜2 hours) underwent volume expansion to ˜1500 μm³ for both the cytoplasm and the nucleus, while the rate of cell volume expansion became slower as local gel deposition increased. The rate of nuclear volume expansion was more sensitive to varied gel deposition than the rate of cytoplasmic volume expansion. Similar effects were also observed with alginate conjugated to a CD44-binding peptide (A5G27; Hibino, et al. (2004) Cancer Res. 64:4810), indicating that the effects are generalizable to other adhesion ligands. In contrast to cell volume expansion by cell spreading as observed in degradable (Khetan, et al. (2013) Nat. Mat. 12:458) or fast stress relaxing (Lee, et al. (2019) Nat. Comm. 10:529) 3D gels, cell volume expansion in alginate-RGD gels was isotropic, as MSCs remained mostly spherical over time. Nearly all MSCs remained within the gel over 3 days. The location of gel-coated MSCs along the z-depth (0-450 μm) did not impact cytoplasmic or nuclear volume regardless of varied gel deposition. Thus, tunable local 3D gel deposition with an adhesion ligand can be used to control the rate of isotropic single cell volume expansion.

As cells expand in volume, stress would be placed on the surrounding gel. Since cell volume expansion in engineered gel deposition was isotropic, it was possible to abstract the system into simple components and derive an analytical solution to calculate the stress on the inner gel surface (σ_{gel}) with a given E when an encapsulated cell with the radius r₁ expands radially by u₀ in response to an adhesion ligand. The analytical solution of the corresponding linear elasticity problem indicated that when the gel was incompressible (Poisson's ratio, v=0.5), σ_{gel} could be expressed as a function of the gel thickness (d_{gel}),

$${σ}_{gel} = \frac{E {u}_{o}}{{r}_{1}} ( \frac{4 {r}_{1}^{3}}{3 {( {r}_{1} + {d}_{gel} )}^{3}} + \frac{2}{3} )$$

Given the constant E, r₁, and u₀, σ_{gel} was increased with thinner d_{gel}, a trend that was also observed if the gel was extendable (i.e., 0≤v≤0.5). The analysis further showed that if the gel was extendable, the gel volume was expected to increase as a result of cell volume expansion, and hence the E of the gel would likely decrease due to reduced polymer density. However, the atomic force microscopy (AFM) analysis showed that E of the outer gel surface remained unchanged when MSCs were encapsulated in the thin gel in the presence or absence of RGD, with or without cell volume expansion, respectively. Hence, the gel composed of 1% w/v 240 kDa alginate was likely close to incompressible. Large strain finite element analysis yielded similar results as the analytical solution, and also showed that the stress on the gel was the highest at the cell-matrix interface. Thus, the gel thickness was an important determinant of the gel stress exerted during isotropic cell volume expansion.

To test whether cells interpret differences in gel stress and subsequently tune their membrane tension as a function of varied gel deposition, a fluorescent lipid tension reporter that changes fluorescent lifetime (τ) upon molecular twisting in response to cell membrane tension (Colom, et al. (2018) Nat. Chem. 10:1118) was used. Reporter activity was measured by two-photon fluorescence lifetime imaging (FLIM). Since the reporter is a small molecule, it readily diffused into alginate gels, which were ˜5 nm in pore size (Boontheekul, et al. (2005) Biomaterials 26:2455), and labeled the membrane of encapsulated MSCs. As a positive control, the relationship between cortical tension (measured by evaluating E using AFM) and membrane tension (measured by τ after seeding cells on 2D poly(ethylene glycol) diacrylate (PEGDA) hydrogels conjugated to RGD with varied elasticity) was assessed. This analysis indicated that both E and τ of cells increased when substrate elasticity increased. In particular, plotting these two parameters showed that E scaled with τ with power law exponent (α) ˜1.8. Results showed that MSCs in the thin alginate-RGD gel exhibited significantly higher τ than MSCs in thicker gels. Thus, tunable local 3D gel deposition with an adhesion ligand can be used to control the membrane tension of single cells independently of local matrix E.

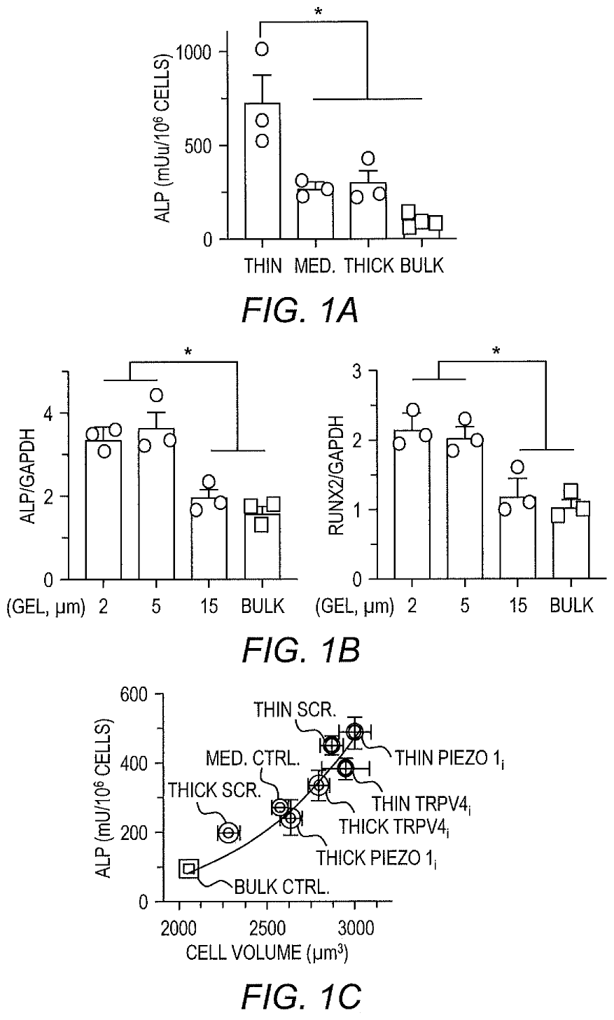

Greater cell volume expansion (Lee, et al. (2019) Nat. Comm. 10:529) or intracellular tension (Engler, et al. (2006) Cell 126:677; Khetan, et al. (2013) Nat. Mat. 12:458; Guo, et al. (2017) Proc. Natl. Acad. Sci. USA 114:E8618) have been reported to skew commitment of MSCs toward osteogenic lineages. Since both phenotypes were observed with varied gel deposition, it was determined whether tuning microscale gel deposition alone was sufficient to influence MSC differentiation. After culturing alginate-RGD gel-encapsulated MSCs for 7 days in medium containing an osteogenesis-promoting cocktail, alkaline phosphatase (ALP) activity was measured to quantify early osteogenic commitment. Strikingly, ALP activity increased as gel deposition became thinner even when the gel E remained at ˜2 kPa (FIG. 1A). To test whether these results reflected osteogenic commitment of multipotent MSCs, gel-coated MSCs were cultured for 10 days in the presence of both osteogenesis and adipogenesis-promoting cocktails. While MSCs in the thin gel showed higher gene expression levels of osteogenic markers, including alp and runx2, MSCs in the thick gel and the bulk gel showed a higher level of an adipogenic marker, pparg1 (FIG. 1B). The diffusivity of small molecules that promote MSC differentiation was less likely impacted by varied gel deposition, since the diffusion kinetics of fluorescein (FITC)-dextran (˜20 kDa) into the gel remained unchanged. Thus, varied local 3D gel deposition with an adhesion ligand impacted the lineage specification of single MSCs.

To establish the causality between isotropic cell volume expansion and membrane tension or osteogenic differentiation regulated by varied local gel deposition, the activity of mechanosensitive ion channels, including Piezol and transient receptor potential vanilloid 4 (TRPV4), was modulated since these channels play roles in cell volume regulation (Hoffmann, et al. (2009) Physiol. Rev. 89:193). Activation of some ion channels is known to drive cell shrinkage by water efflux (Cahalan, et al. (2015) eLife 4:e07370). Treatment of MSCs in the thin alginate-RGD gel with GSK1016790A (GSK101, TRPV4-selective agonist) for 2 hours after encapsulation reduced both cytoplasmic and nuclear volumes. In contrast, treatment of MSCs in the thick gel with GsMTx-4 (inhibitor of some mechanosensitive ion channels, including the Piezo family) or HC-067047 (selective TRPV4 inhibitor) increased both cytoplasmic and nuclear volumes. As expected, GSK101 reduced membrane tension in the thin gel, while GsMTx-4 or HC-067047 increased membrane tension in the thick gel. Consequently, membrane tension directly correlated with cell volume.

To reduce any potential non-specific effects by prolonged treatment of ion channel modulators during MSC differentiation, MSCs were treated with small interfering RNA (siRNA) against Piezol or TRPV4 prior to encapsulation, which lead to a ˜70% decrease in target gene expression. The knockdown of either Piezol or TRPV4 did not impact cell volume in the thin alginate-RGD gel compared to the scrambled control. While the knockdown of TRPV4 accelerated the expansion of both cytoplasmic and nuclear volumes in the thick gel, the knockdown of Piezol failed to increase nuclear volume, indicating that Piezol and TRPV4 distinctly impact volume expansion of MSCs in local gel deposition. As expected, the knockdown of either Piezol or TRPV4 did not impact ALP activity in the thin gel. While Piezol siRNA failed to rescue ALP activity, TRPV4 siRNA rescued ALP activity in the thick gel, indicating that isotropic volume expansion of both the cytoplasm and the nucleus are likely required to promote osteogenic differentiation. The results collectively show that ALP activity scales with cell volume with the power low exponent α˜4.6 (FIG. 1C).

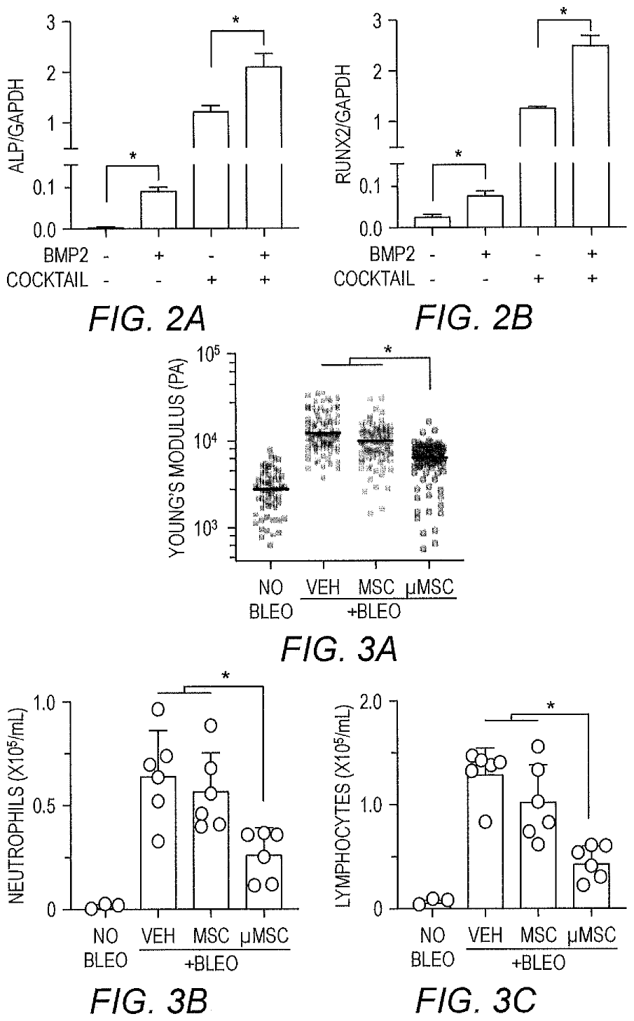

To further demonstrate the osteogenic potential of MSCs encapsulated in RGD-alginate gel coating (5 μm thickness), mouse MSCs were encapsulated in RGD-alginate gel coating with or without embedding a recombinant BMP2 protein. After culturing the alginate-RGD gel-encapsulated MSCs for 7 days in medium with or without an osteogenesis-promoting cocktail, the mRNA expression of osteogenesis markers, alp (FIG. 2A) and runx2 (FIG. 2B) was evaluated by qPCR. Further, use of alginate-RGD gel-encapsulated MSCs in a mouse model of bone marrow ablation demonstrated that thin (5 μm) gel-coated MSCs accelerated bone regeneration one week after treatment as evidenced by an increase in total bone density (˜0.5×10⁵ a.u. in MSC saline controls compared to 3×10⁵ a.u. in alginate-RGD gel-encapsulated MSCs). These analyses demonstrated osteogenic commitment of the MSCs encapsulated in RGD-alginate gel coating.

It has now been shown that the method disclosed herein can control the microscale deposition of engineered hydrogels around individual cells in a 3D space. In addition, varied gel deposition alone has a profound impact on the rate of isotropic cell volume expansion in the presence of an adhesion ligand, which subsequently regulates membrane tension and stem cell differentiation in a predictable manner. The method described here can be readily expanded to elucidate downstream mechanisms behind how single cells respond to engineered local matrix deposition, and how gene expression governing cell fate decision and long-term lineage differentiation is subsequently altered in a distinct manner from elastic modulus and viscoelasticity. The method can also be adapted to be combined with single cell sequencing technologies, in order to understand single cell heterogeneity in biophysical cell-matrix interactions. In engineering cells for regeneration of rigid tissues such as bone, these findings indicate a practical strategy to augment the osteogenic potential of donor MSCs by using a minimal amount of materials, thereby reducing the risk of foreign body reaction and the cost of materials.

### EXAMPLE 2: ALGINATE-COATED MESENCHYMAL STROMAL CELLS FOR TREATMENT OF FIBROSIS

Cell Culture. Clonally derived D1 mouse MSCs of bone marrow origin were purchased from American Type Cell Culture (ATCC). Primary mouse bone marrow MSCs were derived from a C57BL/6J mouse (Cyagen, 8-week-old male). Primary human bone marrow MSCs were derived by plastic adherence of mononucleated cells from a human bone marrow aspirate donor (Lonza, 28-year-old male). All cells were cultured in 37° C., 5% CO₂. D1 and primary mouse MSCs were cultured in complete media composed of high-glucose Dulbecco's Modified Eagle Medium (DMEM; Thermo) supplemented with 10% volume/volume (v/v) fetal bovine serum (FBS; Atlanta Biologicals), 100 units/ml penicillin-100 μg/ml streptomycin, 2 mM GlutaMAX (Thermo). Human MSCs were cultured in α-minimal essential medium (αMEM; Thermo) supplemented with 20% v/v FBS, 100 units/ml penicillin-100 μg/ml streptomycin, and 2 mM GlutaMAX. Cells were passaged when they reached ˜80% confluence in a 175 cm² flask by detaching with trypsin-EDTA (Thermo). D1 mouse MSCs with passage number less than 13, primary mouse MSCs with passage number less than 10, and primary human MSCs with passage number less than 4 were used for this study.

Soluble Inflammatory Mediators. The following soluble inflammatory mediators were used to stimulate cells: Tumor necrosis factor-α (TNFα; 100 ng/ml), Interferon-γ (IFNγ; 100 ng/ml), and Interleukin-1β (IL1β; 100 ng/ml) all purchased from Peprotech. Lipopolysaccharide (LPS; from E. coli O111:B4; 2000 ng/ml) was purchased from Sigma.

Inhibitors of Signaling Pathways. The following chemical inhibitors were used to inhibit signaling pathways while cells were stimulated with TNFα or IL1β for 3 days: SB203580 (10 μM) to inhibit p38 MAPK, SP600125 (20 μM) to inhibit JNK, and U0126 (5 μM) to inhibit ERK1/2, all purchased from Cayman Chemical. The stock solutions of the inhibitors were dissolved in DMSO (Sigma) at 10 mM. DMSO (1:500 dilution, or 28 mM) was used as a control.

Alginate-RGD Preparation. Sodium alginate with ˜240 kDa molecular weight (LF200) was purchased from FMC Biopolymer. An integrin-binding peptide including the sequence Arg-Gly-Asp (GGGGRGDSP (SEQ ID NO:1); Peptide 2.0) was covalently conjugated to alginate by 1-ethyl-dimethylaminopropyl (EDC) and N-hydroxysulfosuccinimide (Sulfo-NHS) (Thermo) chemistry with ˜60 μM according to established methods (Rowley, et al. (1999) Biomaterials 98:184). After conjugation, alginate-RGD was dialyzed against decreasing concentrations of NaCl, charcoal-treated, filter-sterilized, and lyophilized. Lyophilized alginate was stored in −20° C. and dissolved in DMEM within 1 week prior to experiments. The amount of RGD conjugated to alginate was quantified by the LavaPep kit (Gel Company) as described (Ingavle, et al. (2019) Biomaterials 197:119), which is based on a naturally-occurring fluorescent compound that binds to lysine, arginine and histidine residues in peptides (Bell & Karuso (2003) J. Am. Chem. Soc. 125:9304). After reacting the compound with RGD standard solutions dissolved in unconjugated 1% w/v (41.7 μM) LF200 alginate and 1% w/v alginate-RGD samples for 1 hour at 37° C., fluorescent signals were acquired by FlexStation 3 (Molecular Devices) at excitation/emission=540/630 nm. To visualize alginate gels by fluorescence, a small amount (final w/v=0.05% or 4.17 μM) of 10/60 alginate (˜120 kDa; FMC Biopolymer) coupled with Lissamine™ Rhodamine B Ethylenediamine (Thermo) was added prior to gel formation.

Cell Encapsulation in Bulk Alginate-RGD Hydrogels. Cells were resuspended in 1% w/v (41.7 μM) LF200 alginate-RGD in DMEM, and rapidly mixed with calcium sulfate by syringes. Ten or 30 mM final concentration of calcium sulfate (Sigma) was used to form the soft or stiff bulk hydrogel, respectively. The mixed solution was deposited between two glass plates with 1-mm thickness. After 1.5 hours, hydrogels were punched into 5-mm diameter discs and cultured in a 96-well glass bottom plate (MekTak) in the complete media.

Microfluidic Device Fabrication. Microfluidic devices were fabricated using soft lithography. To develop a photoresist, SU-8 3025 (MicroChem) was deposited onto a silica wafer to a defined height and cured by UV light exposure through a transparency mask (CAD/Art Services) for patterning. PDMS (Dow Corning) was then mixed with cross-linker at ratio 10:1, degassed, poured, and cured for at least 3 hours at 65° C. The cured PDMS was peeled off the wafer and bonded to a glass slide by oxygen-plasma treatment of both surfaces. Microfluidic channels were then treated with AQUAPEL® (PPG Industries) and dried. Polyethylene tubing (inner diameter: 0.38 mm; outer diameter 1.09 mm) and 27G×½ needles were used to connect microfluidic channels to syringes (Becton Dickinson).

Single Cell Encapsulation in Alginate Gel Coating. As described previously (Mao, et al. (2017) Nat. Mat. 16:236), CaCO₃ nanoparticles (CalEssence; 900 nm diameter) were resuspended in complete DMEM and dispersed by sonication with a Vibra Cell Sonicator at 75% amplitude for 1 minute. The nanoparticles were then centrifuged at 50 g for 5 minutes to discard larger aggregates, followed by 1000 g for 5 minutes for collection. Purified CaCO₃ nanoparticles were resuspended at 9.6 mg/ml with serum-free DMEM media. Cells were then incubated with CaCO₃ by rotation at room temperature for 1 hour. Excess CaCO₃ nanoparticles were then washed out by centrifugation. The aqueous phase was prepared by resuspending CaCO₃-coated cells in the buffer composed of DMEM with 50 mM HEPES, 10% v/v FBS, 100 units/ml penicillin-100 μg/ml streptomycin at pH 7.4, and mixing cells with 1% w/v (41.7 μM) LF200 alginate-RGD solution. The oil phase included fluorinated oil (3M) with 13 mM perfluoropolyether (PFPE, Miller Stephenson) as a surfactant and 5.3 mM acetic acid (Thermo) as an initiator of Ca²⁺ release from CaCO₃. The aqueous and oil phases were injected into the droplet microfluidic device. The device with channel height 15 μm and width 20 μm was used. The flow rate of the aqueous phase was set to 1 μl/min, while the flow rate of the oil phase was set to 3 μl/min. Emulsion was collected every 20 minutes followed by a 40-minute rotation at room temperature. Emulsion was then broken by the addition of 453 mM 1H,1H,2H,2H-perfluoroctanol (Alfa Aesar). Gel-coated cells were washed twice with serum-free DMEM prior to downstream experiments. Alginate-RGD microgels without cells (˜20 μm in diameter) were synthesized using an aqueous phase composed of 1% w/v alginate-RGD solution mixed with 4.8 mg/ml CaCO₃.

Mechanical Characterization of Hydrogels by AFM. To determine Young's modulus (E) of bulk alginate hydrogels, a gel disc of 5 mm×1 mm was first placed onto a polydimethylsiloxane (PDMS) mold on a glass slide and immersed in a drop of DMEM. To determine E of alginate gel coating after single cell encapsulation, gel-coated cells were immobilized on a glass side pre-coated with 0.1 mg/ml of poly-L-lysine for 2 hours. The sample was then placed in an MFP-3D-BIO system (Asylum Research) to perform indentation analysis with a silicon nitride cantilever with an 18° pyramidal tip (MLCT, Bruker). A spring constant of the cantilever was determined from thermal fluctuations at room temperature (80˜100 mN/m) before each analysis. Fluorescent microscope was used to bring the cantilever to gel coating surface. Indentation was then performed in a contact mode with 500 nm force distance and 1 μm/s velocity and until trigger voltage (0.5 V) was reached, followed by retraction. To calculate E, force-indentation curves were fitted to the Hertzian model and Poisson's ratio=0.5 by using MFP-3D software (14.48.159, Asylum Research).

Characterization of TNFα Diffusion and Retention in Gels. One soft or stiff bulk alginate gel disc (5 mm diameter×1 mm height) or 100,000 soft alginate microgels (˜20 μm diameter) without encapsulated cells were added to each well in a 96-well plate. To measure the extent of TNFα diffusion into gels, each sample was incubated in Fluorobrite DMEM (Thermo) with 100 ng/ml murine TNFα for 8 or 24 hours, followed by washout with DMEM and digestion with alginate lyase. To measure the extent of TNFα retention in gels, each sample was incubated with 100 ng/ml murine TNFα for 24 hours, followed by washout and incubation without TNFα for 0, 24 or 72 hours; at each time point, gels were washed and digested. Murine TNFα ELISA kit (Peprotech) was used to quantify the amount of TNFα in gel digests and total TNFα input against the standard curve.

Confocal Imaging to Measure Cell and Gel Coating Volumes. Cells in gels were incubated with 2 μM calcein AM for 1 hour to stain cytoplasm. Samples were then washed with HBSS and maintained in Fluorobrite DMEM at 37° C. 5% CO₂ during confocal imaging in the Zeiss LSM 770 system with a motorized stage and the 20×/0.8 M27 Plan-Apochromat objective. To analyze cell and gel volume, z-stack images were captured at excitation/emission 495/515 nm and 560/580 nm, respectively, along 60-90 μm in total depth, 0.77 μm each step. IMARIS® (X64 9.3.0, Bitplane) was used to perform 3D reconstruction of images from each stack. Voxels were generated in red and green signals to represent gel and cytoplasm, respectively. Thresholding values were set automatically, showing variations less than 10% across all the images from different experiments.

Retrieval of Cells from Gels. Cells in alginate gels were retrieved by digesting with 3.4 mg/ml alginate lyase (Sigma) at 37° C. for 30 minutes. Samples were then centrifuged at 846×g for 5 minutes and washed twice with HBSS, followed by downstream analyses.

Cell Viability Analysis by Flow Cytometry. Cells were added to the stain buffer composed of HBSS with 2 μM calcein AM (Biotium) and 2 μM ethidium Homodimer-1 (Thermo) for 20 minutes. BD LSRFORTESSA® (Becton Dickinson) was used to acquire fluorescence signals of samples, followed by analysis with the software Weasel (Chromocyte). Percent cell viability was calculated by dividing the number of calcein⁺ ethidium⁻ events by the total event number. In some cases, APC beads (Calibrite™; Becton Dickinson) with a known number were added in each sample to calculate an absolute number of viable and dead cells.

Measurement of Protein Phosphorylation by Intracellular Flow Cytometry. D1 mouse MSCs (100,000 cells) in suspension, soft or stiff alginate-RGD bulk gel were stimulated with either recombinant TNFα or IL1β (100 ng/ml each) for 20 minutes. For MSCs in suspension, the LIVE/DEAD™ fixable violet dead cell stain, for 405 nm excitation (1:1000 dilution, Thermo), was added 10 minutes after stimulation. For MSCs in bulk gels, each gel was rapidly (˜1 minute) digested with 25 mM EDTA with the violet dye in DMEM, followed by incubation of the digested solutions on ice for 7 minutes. Cells were then washed out with DMEM with 0.1% bovine serum albumin (BSA) and fixed with 4% w/v (1.34M) paraformaldehyde in HBSS at room temperature for 10 minutes. After washing twice with HBSS/0.1% BSA, cells were stained with either phospho-p38 MAPK (Thr180/Tyr182) (clone: D3F9) or phospho-JNK (Thr183/Tyr185) (clone: 81E11) rabbit monoclonal antibody (both from Cell Signaling Technology) at 1:100 dilution in the staining buffer (HBSS/0.1% saponin/0.1% BSA) for 2 hours at room temperature. The samples were then washed out once with the staining buffer and incubated with the secondary antibody (donkey anti-rabbit IgG conjugated to the fluorescent dye sold under the trademark ALEXA FLUOR® 546; Thermo) at 1:400 dilution for 40 minutes at room temperature, followed by washing out and resuspension in HBSS. Flow cytometry analysis was done using BD LSRFORTESSA® (Becton Dickinson). The sample incubated with the isotype control (rabbit immunoglobulin G; Cell Signaling Technology) was used as a negative control. Signals from live cell (LIVE/DEAD™ fixable violet dead cell stain negative) fractions were used for analysis. Median fluorescence intensity values of the fluorescent dye sold under the trademark ALEXA FLUOR® 546 were then used to quantify phosphorylated proteins relative to unstimulated samples.

Gene Expression Analysis. Cells were lysed with 1 ml of acid-guanidinium-phenol based reagent sold under the tradename TRIZOL® (Thermo) for 10 minutes. Samples in TRIZOL® were stored at −80° C. if not processed immediately up to 1 week. Chloroform (200 μl) was added per 1 ml of TRIZOL® for phase separation. Samples were centrifuged for 15 minutes at 14,686×g, 4° C. The top layer containing RNA was collected into a new tube, and then precipitated with 250 μl of isopropanol, and 250 μl of 0.8 M sodium citrate combined with 1.2 M sodium chloride for at least 15 minutes at 4° C. Samples were then centrifuged at 14,686×g for 15 minutes at 4° C. The supernatant was removed, and the precipitated RNA was washed with 75% v/v (12.8 M) ethanol (Thermo), followed by centrifugation for 5 minutes at 5287×g, 4° C. After removing ethanol, purified RNA was resuspended in 15 μl of RNase-free water (Thermo). NanoDrop spectrophotometer (Thermo) was used to quantify RNA concentration and quality. cDNA was obtained by reverse transcription using SUPERSCRIPT®-III reverse transcriptase (Thermo). For each sample, 50 ng cDNA was added to each well in triplicate, followed by the Power SYBR Green PCR Master Mix (Thermo). qPCR was performed in the ViiA7 qPCR system (Thermo) with QuantStudio 6/7 software (v1.3). Relative gene expression was calculated using the ΔCt method by comparing each cycle threshold (Ct) value to the reference gene (mouse gapdh or human GAPDH). Primer sequences are presented in Table 4.

TABLE 4

| Gene | | | SEQ ID |
|---|---|---|---|
| (Accession No. | Primer | Sequence | NO: |
| Mouse gapdh | Forward | CTTTGTCAAGCTCATTTCCTGG | 3 |
| (NM_008084) | Reverse | TCTTGCTCAGTGTCCTTGC | 4 |
| Mouse mmp1a | Forward | CCAGTTAAACTTGACGCTGC | 11 |
| (NM_032006) | Reverse | GAAACTGTGGATGTCTCTGGG | 12 |
| Mouse mmp2 | Forward | ACCAAGAACTTCCGATTATCCC | 13 |
| (NM_008610) | Reverse | CAGTACCAGIGTCAGTATCAGC | 14 |
| Mouse mmp13 | Forward | TTGATGCCATTACCAGTCTCC | 15 |
| (NM_008607) | Reverse | ACATGGTTGGGAAGTTCTGG | 16 |
| Mouse acta2 | Forward | GTGAAGAGGAAGACAGCACAG | 17 |
| (NM_007392) | Reverse | GCCCATTCCAACCATTACTCC | 18 |
| Mouse actb | Forward | ACCTTCTACAATGAGCTGCG | 19 |
| (NM_007393.5) | Reverse | CTGGATGGCTACGTACATGG | 20 |
| Mouse tgfb1 | Forward | CCTGAGTGGCTGTCTTTTGA | 21 |
| (NM_011577.2) | Reverse | CGTGGAGTTTGTTATCTTTGCTG | 22 |
| Human GAPDH | Forward | ACATCGCTCAGACACCATG | 23 |
| (NM_002046) | Reverse | TGTAGTTGAGGTCAATGAAGGG | 24 |
| Human MMP1 | Forward | GCACAAATCCCTTCTACCCG | 25 |
| (NM_002421) | Reverse | TGAACAGCCCAGTACTTATTCC | 26 |
| Human MMP2 | Forward | ACCCATTTACACCTACACCAAG | 27 |
| (NM_001127891) | Reverse | TGTTTGCAGATCTCAGGAGTG | 28 |
| Human MMP13 | Forward | GATGACGATGTACAAGGGATCC | 29 |
| (NM_002427) | Reverse | ACTGGTAATGGCATCAAGGG | 30 |

Quantification of Collagen Degradation in vitro. To test collagenase activity in the conditioned media, 50,000 gel-coated D1 mouse MSCs were cultured in 200 μl complete medium±100 ng/ml murine TNFα for 3 days at 37° C. Each conditioned medium (100 μl) was then mixed with 100 μg/ml DQ-collagen type I (Thermo) that generates fluorescence upon degradation and incubated for 1 day at 37° C.±1 mM 4-aminophenylmercuric acetate (APMA, Sigma). Fluorescent signals were then acquired by PHERAstar® microplate reader (BMG LABTECH) with excitation/emission=488/520 nm. To visualize collagen degradation relative to cells, gel-coated or uncoated D1 mouse MSCs were embedded in 1.25 mg/ml collagen-I matrix containing 100 μg/ml DQ-collagen and cultured in complete DMEM medium±100 ng/ml murine TNFα overnight. One mM APMA was added to each sample to completely activate latent MMPs. In some samples, the pan-MMP inhibitor GM6001 (EMD Millipore, 10 μM) was added to test whether collagen degradation was attributed to MMPs. The fluorescent signal from DQ-collagen degradation was captured by Zeiss LSM 770 confocal microscope under 20×/0.8 M27 Plan-Apochromat objective at excitation/emission 495/515 nm. Z-stack images were captured along 40 μm in total depth, 0.77 μm each step. IMARIS® was used for 3D reconstruction of z-stack images, followed by quantification of total fluorescent volumes per field of view.

Production of Recombinant Protein-Loaded Microgels. The aqueous phase was prepared by mixing catalytically-active recombinant mouse MMP13 protein (105-472aa, 46.5 kDa from E. coli; Lifeome) in 1% w/v LF200 alginate-RGD solution with 4.8 mg/ml CaCO₃ to form MMP13-loaded microgels via the droplet microfluidic device as described herein. The recombinant murine TNFα protein was loaded to either empty or MMP13-loaded microgels by incubating 50 ng/ml TNFα in DMEM with microgels for 30 minutes to load ˜6 ng/ml TNFα, since ˜12.5% of TNFα is loaded in ˜0.4 hour. The amount of MMP13 protein released from microgels was quantified by incubating 50,000 MMP13-loaded microgels per 200 μl complete DMEM medium, followed by collecting the media at 4, 24 and 72 hours. In parallel, 50,000 gel-coated MSCs per 200 μl complete DMEM medium were incubated±100 ng/ml murine TNFα, and the media were collected at 24 and 72 hours. Mouse MMP13 ELISA kit (Lifeome) was used to quantify the amount of MMP13 protein per microgel or cell. Collagen degradation activity of the conditioned media from MMP13-loaded microgels was measured after incubating 50,000 gels per 200 μl media for 1 day by DQ-collagen assay as described herein. MMP13 ELISA and DQ-collagen assays were used to titrate the initial amount of MMP13 mixed in the alginate-RGD solution so that the maximum amount of MMP13 released per microgel would be similar to the maximum amount of MMP13 protein released per TNFα-treated, gel-coated D1 mouse MSC—this amount was determined to be 0.75 μg MMP13 protein per 200 μl alginate-RGD solution to achieve ˜5 fg MMP13 released per microgel.

Animal Model of Bleomycin-Induced Lung Injury. All animal experiments were performed in compliance with NIH and institutional guidelines approved by the ethical committee from the University of Illinois at Chicago. C57BL/6J and B6;129S-Tnf^{tm1Gk1}/J; TNFα^{-/-}) mice were purchased from the Jackson Laboratory. 8- to 12-wk-old mice were anesthetized with ketamine/xylazine (50/5 mg/kg), followed by single dose i.t. administration of bleomycin (0.015 U per 20 g mouse) as described previously (Rajasekaran, et al. (2012) PLoS ONE 7:e41611). Bleomycin was diluted in 30 μl PBS and instilled using a 100 μl pipette tip after exposing the laryngopharynx area of the anesthetized mice. Both nostrils were temporarily closed to facilitate liquid flow into trachea. After 1 or 3 weeks of bleomycin administration, animals received a vehicle (sterile serum-free Fluorobrite DMEM), 100,000 uncoated MSCs, or 100,000 gel-coated MSCs in 50 μl Fluorobrite DMEM per 20 g mouse via the i.t. route as done with bleomycin, or through the i.v. route via retro-orbital injection with a 27G syringe needle. After 1 week of cell delivery, animals were sacrificed for downstream analyses.

Bronchoalveolar Lavage (BAL). One ml of phosphate-buffered saline (PBS, Thermo) with 2 mM ethylenediaminetetraacetic acid (EDTA, Sigma) was added through the trachea and withdrawn by syringe to collect BAL fluid. The BAL fluid was centrifuged at 845×g for 10 minutes at 4° C. Isolated cells were resuspended in 500 μl of PBS with 2 mM EDTA. After counting total cell number by hemocytometer with 99 mM crystal violet (Sigma), 70 μl of cells were deposited onto a glass slide by the CYTOSPIN® centrifuge (Thermo) at 294×g for 5 minutes. Kiwi Diff™ staining kit (Thermo) was used to stain cells on the slide, followed by counting the number of macrophages, lymphocytes and neutrophils.

Quantification of Hydroxyproline. Lung tissue from each mouse was weighed and homogenized in 1 ml of water. One hundred μl of homogenate was mixed with 100 μl of 12M HCl and incubated at 120° C. for 3 hours. An assay kit (Sigma) was used to quantify hydroxyproline. Ten μl of the lysate was loaded per well in a 96-well plate and dried at 65° C. for 1.5 hours. Chloramine T oxidative buffer (100 μl; Sigma) was then added to each well and incubated at room temperature for 5 minutes. 4-(Dimethylamino) benzaldehyde (DMAB, 100 μl; Sigma) was added to each well and incubated for 1.5 hours at 65° C. Colorimetric signals were captured at 560 nm absorbance using a plate reader (PHERAstar® 3.0, BMG LABTECH). The level of hydroxyproline was extrapolated by the standard curve, and the total amount was then calibrated by the weight of the total tissue.

Histology with Masson's Trichrome Stain. The lungs were perfused with PBS as described (Rajasekaran, et al. (2012) PLoS ONE 7:e41611) to clear blood cells prior to histology, followed by fixation in 2% w/v (0.67 M) paraformaldehyde (Sigma) in PBS for 48 hours. Tissues were then embedded into paraffin blocks and sectioned using a microtome with 5-μm thickness. Slides with tissue sections were baked at 45° C. for 2 hours and washed with xylene to remove paraffin, followed by rehydration with gradient ethanol 100% v/v (17 M), 95% (16 M) and 75% (12.8M). Rehydrated samples were then treated with preheated Bouin's solution (Sigma) for 1 hour, followed by Masson's trichrome staining using a kit (Sigma) based on the manufacturer's protocol. The stained slides were dehydrated by gradient ethanol 75%, 95%, 100%, washed with xylene, mounted, and dried for 24 hours. The Aperio ImageScope system (Leica) was used to digitize histology slides. The Orbit Image Analysis software (version 3.15) was used to quantify histology images by machine learning as described (Seger, et al. (2018) PLoS ONE 13:e0193057). The object training function was used to classify fibrotic vs. normal mass at low magnification and fibrotic vs. normal alveoli at high magnification. Ten representative regions from each of normal and bleomycin-treated lung tissue sections were used to train the software. The trained model was then applied to all samples for automated analysis.

Second Harmonic Imaging Microscopy. Imaging was done with second harmonic generation (SHG) in the Ultima Multiphoton Microscope System (Bruker) to quantify collagen fibers and elastin in freshly isolated lung tissue as described (Pena, et al. (2007) Microsc. Res. Tech. 70:162). The Chameleon Ultra II Two-Photon laser (860 nm) operating at 80 MHz was used to excite lung tissue. Collagen fibers were visualized by capturing backward scattering of SHG through a bandpass 430/24 nm filter. Elastin was visualized through a 582/22 nm filter. Z-stack images of collagen and elastin signals were acquired in parenchymal regions (defined between 20 and 50 μm in depth from tissue surface) with 1 μm interval via Prairie View software (5.4, Bruker), followed by 3D reconstruction and quantification of total elastin volume (Eᵥ) collagen volume (Cᵥ) by IMARIS®. The elastin-to-collagen volume ratio index was calculated as described (Lin, et al. (2005) Opt. Lett. 30:2275): (Eᵥ−Cᵥ)/(Eᵥ+Cᵥ).

Quantification of Lung Tissue Microelasticity. After perfusion of the lungs, 800 μl of the optimal cutting temperature compound was added through the trachea. The lungs were then frozen in 4-methyl butane chilled with dry ice and stored at −80° C. for no longer than 1 week. Tissue slices (15 μm) were sectioned with the HM525 MX Cryostat (Thermo) and stored at −20° C. for no longer than 24 hours before analysis. After thawing tissue slices at room temperature for 10 minutes and rinsing them with PBS, AFM was performed to measure tissue microelasticity with 250 nm force distance and 0.5 μm/s tip velocity until trigger voltage (0.5 V) was reached.

RNA Interference. Small interfering RNAs (siRNAs) were purchased from Thermo Fisher Scientific as follows: mmp13 (Assay ID: 155380) and scrambled (Silencer negative control no. 1 siRNA). siRNA (4 nM) was mixed with transfection reagent sold under the tradename LIPOFECTAMINE® RNAiMAX® (Thermo) for 15 minutes in Opti-MEM® medium (Thermo). The mixture was then applied to MSCs and cultured for 1 day prior to gel coating. qPCR was used to confirm the knockdown efficiency of each target gene compared to the scrambled control.

Establishing MSCs that Express Firefly Luciferase. To introduce firefly luciferase in D1 mouse MSCs, premade lentiviral particles containing mCherry-IRES-Firefly were purchased from the Mass General Hospital Vector Core. MSCs were incubated with viral particles for 2 days. Fluorescent MSCs were then isolated by fluorescence-activated cell sorting (FACS) and expanded for further analysis.

Tracking Biodistribution of MSCs in vivo. One week after bleomycin treatment, uncoated or gel-coated firefly luciferase expressing D1 mouse MSCs were delivered via i.t. or i.v. routes. The mice were then i.v. injected with 200 μl of 15 mg/ml D-luciferin (Syd Labs) in PBS per 20 g mouse at different time points, followed by bioluminescence imaging with the IVIS Spectrum (PerkinElmer) within 10 minutes of D-luciferin injection to measure an average radiance (photons/sec/cm²/sr) in the lung region using the Living Image software (4.0, PerkinElmer).

Mathematical Modeling. A set of differential equations were constructed to predict the effect of uncoated or gel-coated MSCs on the total collagen level in lung tissue, and numerically solved by using the ode45 function in MATLAB (R2017a, Mathworks). To simulate the effect of cell delivery 1 week after bleomycin treatment, the following initial values were used: MSCo=100,000 cells; Bleo₀=0.215 U/kg; TNFα₀=0.49 ng/ml; MMP₀=432 pg/ml; Col₀=0.3 μg/mg tissue. To simulate the effect of cell delivery 3 week after bleomycin treatment, the following initial values were used: MSC₀=100,000 cells; Bleo₀=0.0056 U/kg; TNFα₀=0.2 ng/ml; MMP₀432 pg/ml; Col₀=0.6 μg/mg tissue.

Statistical Analysis. Statistics were performed as described in figure captions. All statistical analyses were performed using GraphPad Prism version 8.1.0. Unless otherwise noted, statistical comparisons were made by one-way ANOVA followed by Tukey's multiple comparisons test when standard deviations did not vary between test groups, and by one-way Welch ANOVA followed by Dunnett T3 multiple comparisons test when standard deviations were variable. For the data that do not show normal distribution based on D'Agostino-Pearson normality test, Kruskal-Wallis one-way ANOVA followed by Dunn's multiple comparisons test was used. To compare the mean differences between groups that have been split on two independent variables, two-way ANOVA followed by Bonferroni's multiple comparisons test was used. A p-value less than 0.05 established statistical significance.

Modeling MSC-Mediated Degradation of Collagen by Interstitial Collagenases. A set of differential equations were constructed to model the effect of donor MSCs on collagen levels in lung tissue challenged by bleomycin. A simple equation was used to describe the kinetics of donor MSCs after i.t. delivery as follows:

$$\frac{d M S C}{d t} = ( {β}_{M S C} - {α}_{M S C} ) M S C$$

β_{MSC} is the production (or proliferation) rate, while α_{MSC} is the death rate of donor MSCs. Based on the biodistribution kinetics data from i.t. delivery of MSCs, β_{MSC}=0, and α_{MSC}=0.937/day, which is equivalent to decay t_{1/2}=0.74 day or 17.78 hours.

The kinetics of bleomycin after delivery is described as follows:

$$\frac{dBleo}{d t} = - {α}_{bleo} Bleo$$

α_{bleo} is the clearance rate of bleomycin and set to 0.2505/day or t_{1/2}=2.77 day, starting from 2 day after bleomycin treatment as described^{[58]}.

The kinetics of TNFα in the host is described as follows:

$$\frac{d T N F α}{d t} = {β}_{tnf α} Bleo - {α}_{tnf α} T N F α$$

β_{tnfα} is the production rate of TNFα in the host induced by bleomycin and set to 0.135. α_{tnfα} is the decay rate of TNFα and set to 0.08. These values are based on the previously described kinetics of TNFα when a single dose of bleomycin is administered (Smith, et al. (1998) J. Leukocyte Biol. 64:528).

The kinetics of interstitial collagenases is described as follows:

$$\frac{d M M P}{d t} = {β}_{basal} + {β}_{mmp} {θ}_{tnf α} M S C - {α}_{mmp} M M P$$

β_{basal} is the basal production rate of collagenases by the host and set to 300 pg/ml/day, which is within the previously described range in lung tissue (Ratjen, et al. (2002) Thorax 57:930). Production of collagenases by donor MSCs depends on two factors: 1. βₘₘₚ: the maximum production rate of collagenases from donor MSCs (varied during simulation from 0.4 to 4.0); 2. θ_{tnfα}: the dose response of TNFα to activate TNFα receptors on donor MSCs:

$${θ}_{t𝔫fα} = \frac{1}{1 + {( \frac{{K}_{tnf α}}{T N F α} )}^{2}}$$

where K_{tnfα} is TNFα concentration to achieve the half-maximum activation of TNFα receptors and set to 1 ng/ml as described (Turner, et al. (2010) J. Cell Sci. 123:2834). αₘₘₚ is the degradation rate of collagenases, for instance, by host TIMPs, and set to 0.693/day or t_{1/2}=1 day as described (Urbach, et al. (2015) Chem. Biol. 22:1442).

The kinetics of total collagen in lung tissue is described as follows:

$$\frac{dCol}{d t} = {β}_{col} Bleo - δ ( Col )$$

β_{col} is the maximum production rate of collagen induced by bleomycin (Bleo) and set to 0.22 μg/mg tissue/day starting 1 week after instillation of bleomycin as described (Izbicki, et al. (2002) Int. J. Exp. Pathol. 83:111). δ(Col) is the rate of collagen degradation based on the Michaelis-Menten equation:

$$δ ( Col ) = {V}_{max} \frac{{Col}^{2}}{{Col}^{2} + {{K}_{col}}^{2}} = \frac{{α}_{col} M M P}{1 + {( \frac{{K}_{col}}{Col} )}^{2}}$$

α_{col} is the catalytic rate constant of interstitial collagenases and set to 0.02/day or t_{1/2}=34.67 day as described (Han, et al. (2010) J. Biol. Chem. 285:22276). K_{col} is the concentration of collagen at the half-maximum rate of degradation and set to 25 μg/mg tissue or ˜83 μM collagen (assuming tissue density ˜1 mg/μl and molar mass of collagen=300 kDa), which is close to the previously described range for MMP1 and MMP13 (Solomonov, et al. (2016) Proc. Natl. Acad. Sci. USA 113:10884).

Therapeutic Efficacy of Gel-Coated MSCs to Inhibit Muscle Fibrosis. It has been shown that fibrotic phenotypes in skeletal muscles of D2.mdx mice are observed as early as 4 weeks of age and become more severe at 7 weeks of age and afterwards (van Putten, et al. (2019) FASEB J. 33:8110; Heydemann, et al. (2009) J. Clin. Invest. 119; 3703; Coley, et al. (2016) Hum. Mol. Genet. 25:130; Mazala, et al. (2020) JCI Insight 5(6): e135703). The effect of gel-coated MSCs on D2.mdx mice delivered at different ages: 4, 7 and 24 weeks is tested using the experimental groups described in Table 5.

TABLE 5

| Strain | Group | Description |
|---|---|---|
| DBA | 1 | PBS |
| D2.mdx | 2 | PBS |
| | 3 | Empty gel |
| | 4 | Empty gel + TNFα |
| | 5 | Uncoated muscle MSCs |
| | 6 | Uncoated muscle MSCs + TNFα |
| | 7 | Muscle MSCs in thin (5 μm) gel |
| | 8 | Muscle MSCs in thin gel + TNFα |
| | 9 | Uncoated marrow MSCs |
| | 10 | Uncoated marrow MSCs + TNFα |
| | 11 | Marrow MSCs in thin (5 μm) gel |
| | 12 | Marrow MSCs in thin gel + TNFα |

Both primary muscle and marrow MSCs are isolated from DBA mice and used in these studies. Uncoated vs. thin (5 μm) alginate-RGD gel-coated MSCs are compared. D2.mdx mice are known to show a variable level of endogenous TNFα in skeletal muscles where it increases from week 4 and peaks at week 8, followed by gradual decrease afterwards (Hammers, et al. (2020) Sci. Rep. 10:14070). Thus, experimental groups are included where uncoated MSCs are treated with recombinant murine TNFα (100 ng/ml) for 1 day, or the gel coating of MSCs incorporates TNFα. Empty microgels with the compatible size as gel-coated MSCs (˜28 μm in diameter)±TNFα are included as controls. To increase the chance of observing improved functional outcomes, treatment is carried out in both gastrocnemius muscles, 100,000 cells or gels per muscle of 20 g D2.mdx mouse. After delivery, skeletal muscle functions are tested in terms of hindlimb grip strength and hanging time, with measurements taken for 4 weeks, twice a week. Body mass is measured as well. To test whether improved functional outcomes are associated with reduced muscle fibrosis, a selected group of mice are sacrificed at a time point where functional improvement by gel-coated MSCs with or without TNFα can be seen (e.g., 2 weeks after injection). Collagen fibers are quantified by second harmonic imaging using two-photon microscopy. The results are corroborated by histological analysis to quantify collagen by Trichrome staining and muscle fiber diameter by reticulin staining (van Putten, et al. (2019) FASEB J. 33:8110), followed by quantification in an unbiased manner by writing a MATLAB code (Gilhodes, et al. (2017) PLoS ONE 12:e0170561). qPCR analysis of gastrocnemius muscles is carried out to quantify mRNA expression of genes associated with inflammation (e.g., TNFα, Il1β), matrix remodeling (e.g., mmp13, mmp1a, mmp2), fibrosis (e.g., Tgfb1), collagen (e.g., Col1a1) and fat infiltration (e.g., Pparg1).

Biodistribution of Gel-Coated MSCs in Skeletal Muscles of D2.mdx Mice. Biodistribution of donor MSCs after intramuscular injection in D2.mdx mice is evaluated and the effect of gel coating and pre-activation with TNFα is tested. To facilitate live imaging and characterization of donor MSCs in vivo, both primary muscle and marrow MSCs are virally transduced with firefly luciferase and mCherry. The experimental groups are described in Table 5 and delivered at different ages of D2.mdx mice: 4, 7 and 24 weeks. In these studies, 100,000 cells or gels per muscle of 20 g mouse are delivered to one gastrocnemius muscle, while the other muscle is injected with PBS. This experimental design provides for testing whether donor MSCs remain localized within the injected muscle or they traffic into the other muscle. IVIS imaging is done at different time points after injection (1 hour; 1, 4 and 7 days; twice a week after the first week, up to 4 weeks) by looking at firefly luciferase activity. In addition to donor MSCs, gel coatings are also visualized simultaneously by mixing a fraction ( 1/20) of alginate conjugated to fluorescent dye sold under the tradename ALEXA FLUOR® 750 as described (Mao, et al. (2017) Nat. Mater. 16:236). For samples showing long-term presence of MSCs at the end timepoint of live animal tracking, localization of donor MSCs in gastrocnemius muscles is examined by histological analyses after staining frozen sections with F-actin (phalloidin), endothelial (CD31⁺), and mesenchymal (PDGFRα⁺) markers. The distribution of mCherry⁺ donor MSCs against the host cells is then evaluated by confocal microscopy.

Soft Matrix Enhances Soluble Interstitial Collagenase Production in MSCs by TNFα. To demonstrate that MSCs respond to specific inflammatory and matrix biophysical signals, and subsequently correct aberrant tissue remodeling processes via the release of paracrine factors—here, focus was placed on MMPs, which regulate a number of fundamental processes involving normal and aberrant tissue remodeling (Loffek, et al. (2011) Eur. Respir. J. 38:191; Bonnans, et al. (2014) Nat. Rev. Mol. Cell Biol. 15:786). Tumor necrosis factor-α (TNFα), interferon-γ (IFNγ), interleukin-1β (IL1β) and lipopolysaccharide (LPS) were first tested as inflammatory signals, since these molecules have been previously implicated in eliciting anti-inflammatory and therapeutic functions of MSCs as an adaptive response to injury (Le Blan & Mougiakakos (2012) Nat. Rev. Immunol. 12:383). Clonally-derived marrow D1 mouse MSCs were chosen for initial studies, since clonal populations provide greater cell-to-cell homogeneity than primary cells (Mao, et al. (2016) Biomaterials 98:184). This analysis indicated that TNFα upregulated mRNA expression of soluble interstitial collagenases, including mmp13 and mmp1a, to a greater extent than other tested inflammatory signals. In contrast, TNFα did not substantially upregulate gelatinases (e.g., mmp2). Primary mouse and human bone marrow MSCs also showed similar effects. Thus, TNFα is a prominent inducer of soluble interstitial collagenase expression in MSCs.

To identify downstream signaling pathways that mediate TNFα-induced upregulation of soluble interstitial collagenases, mmp13 mRNA expression was measured in response to TNFα in the presence of inhibitors against p38 mitogen-activated protein kinase (MAPK), c-Jun N-terminal kinase (JNK), and extracellular signal-regulated kinase 1/2 (ERK1/2) pathways; SB203580 (10 μM), SP600125 (20 μM), and U0126 (5 μM), respectively. Inhibition of p38 MAPK and JNK but not inhibition of ERK1/2 suppressed TNFα-induced upregulation of mmp13 mRNA. In contrast, only inhibition of p38 MAPK was able to reduce IL1β-induced upregulation of mmp13 mRNA, indicating that JNK is selective to TNFα-induced upregulation of mmp13 mRNA. TNFα induced phosphorylation of JNK isoforms p46 (at Thr183) and p54 (at Tyr185) in 20 minutes to a greater extent than IL1β, while both TNFα and nap induced phosphorylation of p38 MAPK at Thr180 and Tyr 182 to a similar extent. These results indicated that the JNK pathway plays an important role in TNFα-induced mmp13 expression.

The impact of pathophysiologically relevant mechanical cues on the ability of MSCs to express MMPs in response to TNFα was subsequently analyzed. A synthetic alginate (LF200, ˜240 kPa, 1% weight per volume (w/v)) hydrogel system conjugated with the minimal integrin adhesion ligand Arg-Gly-Asp (RGD) (˜60 μM) with tunable stiffness (Young's modulus, E) (‘alginate-RGD’) was used. Most D1 mouse MSCs remained viable 3 days after encapsulation in soft (E˜2 kPa) or stiff (E˜20 kPa) bulk gels. Hydrogel stiffness did not impact the diffusion of TNFα recombinant protein into the gels. To characterize the effect of hydrogel stiffness on TNFα-induced expression of collagenases, dose-response studies were performed. The soft bulk gel increased the maximum response of TNFα to upregulate mmp13 and mmp1a mRNA by ˜1.9-fold and ˜3.0-fold, respectively. In addition, the soft bulk gel reduced the dose of TNFα required for the half-maximum response (i.e., increased potency) to upregulate mmp13 and mmp1a mRNA by ˜4.5-fold and ˜1.3-fold, respectively. Consistently, TNFα-induced phosphorylation of JNK was higher when MSCs were encapsulated in the soft bulk gel than the stiff bulk gel. Thus, TNFα and soft matrix constitute chemomechanical cues that enhance the production of soluble interstitial collagenases by MSCs.

Gel Coating Enables MSCs to Degrade Collagen Over Distance in Response to TNFα. It was determined whether MSCs treated with TNFα in soft matrix could be leveraged to degrade collagen in a paracrine manner, which would be useful to control remodeling processes that result in tissue rigidification by collagen deposition or cross-linking (Halle, et al. (2018) Nano Lett. 18:1). A droplet microfluidic approach was used to miniaturize the soft bulk alginate-RGD gel into a conformal gel coating of individual cells (μMSC). To facilitate delivery to tissues with a narrow space, such as small airways, the volume of gel coating was minimized by 2˜3 times that of a single MSC while maintaining soft E at ˜2 kPa. This approach yielded gel-coated viable MSCs with high efficiency without the need to perform additional cell sorting, and is thus ideally suited for therapeutic uses. A high molecular weight alginate (˜240 kDa) was chosen to keep most MSCs from proliferating (Mao, et al. (2017) Nat. Mater. 16:236), as confirmed by a low level of 5-ethynyl-2′deoxyuridine (EdU) incorporation for 1 day, as opposed to MSCs in collagen-I gel. Importantly, gel coating keeps MSCs from adopting myofibroblast phenotypes for at least 3 days after encapsulation, since the gene expression of α-smooth muscle actin (acta2), beta-actin (actb), and tumor growth factor-μl (tgfβ1) remained lower than MSCs in the stiff bulk gel, consistent with a previous study (Dingal, et al. (2015) Nat. Mater. 14:951); tgfβ1 expression was even lower in gel-coated MSCs than MSCs in the soft bulk gel.

TNFα-induced expression of collagenases in gel-coated MSCs was then tested. Despite the similar diffusion kinetics of TNFα into the microgel versus the bulk gel with the same E˜2 kPa, gel coating of single D1 mouse MSCs resulted in ˜3-times higher TNFα-induced upregulation of mmp13 and mmp1a than bulk gel encapsulation of MSCs. Treatment of gel-coated MSCs with TNFα increased collagenase activity in the conditioned media, which was further enhanced by ˜2-fold when the media were treated with the saturating concentration (1 mM) of 4-aminophenylmercuric acetate (APMA), the chemical activator of latent MMPs (Galazka, et al. (1996) Biochemistry 35:11221). Thus, gel-coated MSCs secrete both active and latent forms of collagenases in response to TNFα. Gel-coated D1 mouse MSCs were capable of degrading the surrounding collagen-I ex vivo upon activation with TNFα for 1 day in culture in an MMP-dependent manner, since incubation with the pan-MMP inhibitor GM6001 (10 μM) suppressed this process. Most MSCs remained within the microgels, indicating that gel-coated MSCs can degrade collagen-I in a paracrine manner. Thus, gel coating is an enabling cue for MSCs to degrade collagen over distance by producing soluble collagenases in response to TNFα.

Gel Coating Enables MSCs to Inhibit Aberrant Remodeling After Fibrotic Lung Injury in vivo. To investigate the therapeutic relevance of these results, it was determined whether gel coating enables MSCs to inhibit tissue rigidification induced by fibrotic injury during acute host inflammation. A bleomycin-induced injury model was used to demonstrate this since it has been widely leveraged to reproducibly recapitulate some aspects of aberrant tissue remodeling, including enhanced collagen production and cross-linking (Liu, et al. (2017) Meth. Mol. Biol. 1627:27). The lungs were chosen as the model organ of interest, since they are frequently exposed to exogenous injury, which could lead to scar formation in interstitial regions, thereby compromising respiration (Thannickal, et al. (2014) J. Clin. Invest. 124:4673). Lung fibrosis is also a terminal disease for afflicted patients due to the lack of efficacious therapies (Mora, et al. (2017) Nat. Rev. Drug Discov. 16:755). To this end, bleomycin (0.015 U per 20 g mouse) was instilled intratracheally (i.t.) to induce injury in the mouse lungs. The basal total collagen level in the lungs was ˜0.3 μg hydroxyproline/mg tissue. After bleomycin treatment, this value remained at the baseline at week 1 but increased by ˜2-fold to ˜0.6 μg/mg at week 2. Infiltration of neutrophils, lymphocytes, and macrophages into the airways also increased and became maximum 2 weeks after bleomycin treatment, indicating a high level of inflammation.

To test whether gel coating enabled MSCs to suppress excess collagen deposition, 100,000 gel-coated D1 mouse MSCs were i.t. delivered per 20 g mouse after 1 week of bleomycin treatment, followed by analyses after 1 week of cell delivery. Gel-coated MSCs, but not uncoated MSCs, significantly limited total hydroxyproline deposition to ˜0.45 μg/mg in the lungs. Automated analyses of Masson's trichrome stained lung sections based on machine learning (Seger, et al. (2018) PLoS ONE 13:e0193057) showed decreased fibrotic mass and increased maintenance of normal alveoli after delivery of gel-coated MSCs compared to other tested groups. Three-dimensional (3D) two-photon analyses of fresh lung samples (Pena, et al. (2007) Microsc. Res. Tech. 70:162) showed that only gel-coated MSCs significantly reduced accumulation of fibrillar collagen in the lung parenchyma. To test whether gel-coated MSCs also preserve physiological lung tissue stiffness against bleomycin treatment, atomic force microscopy (AFM) was used to measure microelasticity of lung parenchymal sections (Liu, et al. (2016) JCI Insight 1:e86987; Liu, et al. (2010) J. Cell Biol. 190:693). Gel-coated MSCs, but not uncoated MSCs, significantly reduced lung tissue stiffness by ˜2.3-fold compared to the vehicle control (FIG. 3A). Gel-coated MSCs were effective in reducing infiltration of neutrophils and lymphocytes into the airways (FIG. 3B and FIG. 3C), while macrophage infiltration remained unchanged. The MSC dose required for achieving therapeutic efficacy was subsequently assessed and it was observed that lower doses of gel-coated MSCs were not effective, thus indicating that 100,000 gel-coated MSCs per 20 g mouse was a minimum effective dose in this context. Increasing the dose of uncoated MSCs to 500,000 cells per 20 g mouse was not sufficient to recapitulate the effect of gel-coated MSCs. The gel alone was not bioactive, since empty alginate-RGD microgels (˜20 μm in diameter) that did not contain cells showed no significant effect when delivered via either i.t. or intravenous (i.v.) route. Gel coating of primary mouse bone marrow MSCs also resulted in reduced levels of collagen deposition, and infiltration of neutrophils and lymphocytes; this effect was observed with both i.t. and i.v. routes of delivery. Thus, gel coating of MSCs is an effective means to suppress fibrotic lung injury while minimizing the number of required therapeutic donor cells.

Delivery Routes Determine the Effect of Gel Coating on the Residence Time of MSCs. To test whether the ability of gel-coated MSCs to inhibit fibrotic lung injury can be attributed to the increased residence time of MSCs, D1 mouse MSCs that express firefly luciferase were used to track their biodistribution kinetics in vivo. Both i.v. and i.t. routes led to localization of MSCs in the lung regions of the mice previously treated with bleomycin for 1 week, within 30 minutes of administration. Consistent with the previous results (Parekkadan & Milwid (2010) Ann. Rev. Biomed. Engin. 12:87), i.v. injected, uncoated MSCs were rapidly cleared from the lungs (t_{1/2}˜5 hours) with no evidence of subsequent integration in other tissues. Gel coating significantly increased the residence time of i.v. injected MSCs by ˜3.5-fold (t_{1/2}˜18 hours) and enhanced the remaining fraction of MSCs by ˜2-fold in 7 days. In contrast, i.t. delivered, uncoated MSCs were cleared slower than i.v. injected, uncoated MSCs by ˜3-fold (t_{1/2}˜15 hours). Despite this, uncoated MSCs did not significantly inhibit bleomycin-induced lung injury. Gel coating did not delay the clearance of i.t. delivered MSCs in terms of half-life, plateau, and total area of the biodistribution curve, thereby indicating that the effect of gel coating on the residence time of MSCs depended on routes of administration. Thus, the ability of gel-coated MSCs to suppress fibrotic lung injury was not due to increased residence time of MSCs.

Donor mmp13 and Host TNFα Determine the Efficacy of Gel-Coated MSCs in Fibrotic Injury. It was determined whether soluble collagenase from gel-coated D1 mouse MSCs contributed to their efficacy in bleomycin-induced lung injury. Mmp13 was chosen as a soluble interstitial collagenase of interest for mechanistic studies, because its expression level is generally much higher than the other isoform mmp1a. D1 mouse MSCs were treated with mmp13 siRNA or scrambled siRNA prior to gel coating. Transfection of siRNA in MSCs led to ˜70% knockdown (k.d.) of mmp13 in 1 day compared to scrambled siRNA. Gel-coated MSCs treated with mmp13 siRNA were no longer able to ameliorate hydroxyproline deposition compared to those treated with scrambled. The reduction of both neutrophils and lymphocytes by gel-coated MSCs was also no longer seen with mmp13 siRNA, indicating that MMP13 suppressed recruitment of these cells into the lung airways. Therefore, MMP13 from donor MSCs is essential for gel-coated MSCs to suppress fibrotic lung injury.

It was subsequently determined whether gel-coated MSCs require host TNFα to suppress aberrant tissue remodeling in bleomycin-induced lung injury. The total collagen level in the lungs of TNFα^{-/-} mice was increased ˜2-fold after 1 week of bleomycin treatment, just like in wild-type mice. However, gel-coated MSCs were no longer able to inhibit hydroxyproline deposition in the bleomycin-treated TNFα^{-/-} mice. Gel-coated MSCs also failed to reduce neutrophil infiltration in these mice, but they could still reduce lymphocyte infiltration independently of TNFα. Treating with recombinant TNFα prior to administration rescued the ability of gel-coated MSCs to reduce hydroxyproline deposition and neutrophil infiltration in TNFα^{-/-} mice. Together, the results showed that both host TNFα and donor MMP13 were required for the ability of gel-coated MSCs to ameliorate fibrotic lung injury.

Predicting the Efficacy of MSCs Delivered at Different Time Points After Fibrotic Lung Injury. A mathematical model was developed to gain quantitative insights into how MSCs influence collagen deposition when delivered at different time points upon tissue injury. In this model, both maximum production rate (βₘₘₚ) and dose response of TNFα binding to MSCs (θ_{tnfα}) determine collagenase secretion from MSCs, while a classical Michaelis-Menten equation describes collagen degradation kinetics by collagenases (δ_{col}). Parameter values were set to recapitulate the phenotypes known to be induced by instillation of single dose bleomycin; TNFα in the airways is transiently increased to maximum at week 1, followed by decrease over 5 weeks (Smith, et al. (1998) J. Leukocyte Biol. 64:528), while tissue collagen is increased to maximum between week 2 and 4, and later decreases at week 6. The model showed that increasing βₘₘₚ as achieved by gel coating was sufficient to inhibit collagen deposition when MSCs were delivered 1 week after bleomycin treatment; setting βₘₘₚ to 1.2 and 0.4 recapitulated the experimental results from delivering gel-coated MSCs and uncoated MSCs, respectively. After 3 weeks of bleomycin treatment when collagen deposition peaked, the host TNFα level became lower from the maximum by 2.5-fold; when MSCs were delivered at this time point, the model indicated that both gel-coated (βₘₘₚ=1.2) and uncoated (βₘₘₚ=0.4) MSCs would not be as effective in reducing established collagen. When the host TNFα level was restored to the maximum, however, the model predicted that gel-coated MSCs would reduce collagen deposition more substantially than uncoated MSCs when delivered at week 3. The sensitivity analysis indicated that increasing βₘₘₚ would decrease the half-maximum TNFα concentration necessary to reduce collagen. Thus, the model predicted the importance of both gel coating and reconstituting TNFα in enabling MSCs to facilitate normal tissue remodeling of injured tissues.

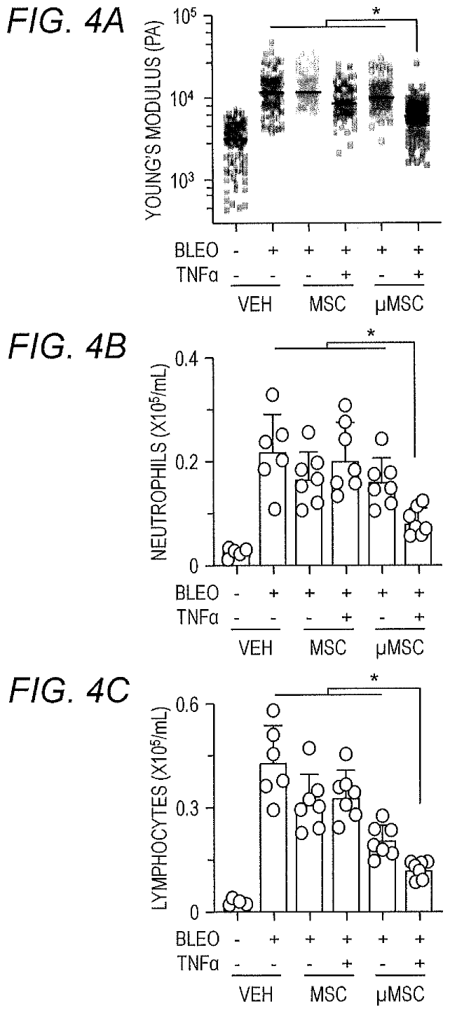

Gel-Coated MSCs Loaded with TNFα Facilitate the Resolution of Fibrotic Phenotypes. A strategy was developed to promote normal tissue remodeling at later time points when fibrosis had set in and inflammation had already subsided. Approximately 10% of total recombinant mouse TNFα protein was stably incorporated in alginate microgels for at least 3 days after incubation with the solution containing TNFα for 1 day, followed by washing out unbound TNFα. This strategy was used to continuously present MSCs with recombinant TNFα in gel coating and to test their efficacy in restoring injured tissues after aberrant remodeling by administering the mice 3 weeks after bleomycin treatment. Consistent with the model, only gel-coated, TNFα-loaded MSCs significantly reduced established collagen to ˜0.4 μg/mg 1 week after delivery. In addition, gel-coated, TNFα-loaded MSCs significantly reduced fibrotic mass, restored normal alveoli, and brought fibrillar collagen in the lung parenchyma to physiological levels. Gel-coated, TNFα-loaded MSCs were also effective in restoring lung tissue closer to the physiological stiffness compared to other groups (FIG. 4A). Gel-coated, TNFα-loaded MSCs accelerated the resolution of neutrophil and lymphocyte infiltrations (FIG. 4A and FIG. 4B, respectively), while macrophage infiltration remained unchanged.

To test whether TNFα or MMP13 was sufficient to resolve fibrotic phenotypes, alginate-RGD microgels (˜20 μm in diameter) were loaded with either recombinant mouse TNFα or catalytically active mouse MMP13 proteins. The diffusion kinetics of TNFα into microgels was fast (t_{1/2}˜0.46 hour), while that of MMP13 from microgels was an order of magnitude slower (t_{1/2}˜4 hours). This feature provided an opportunity to mimic TNFα-loaded, gel-coated MSCs by loading TNFα briefly (˜30 minutes) after the formation of microgels pre-loaded with MMP13, while still allowing microgels to continuously release MMP13 as they were administered in mice. The amount of MMP13 pre-loaded to microgels was chosen so that the maximum level of MMP13 released per microgel would be similar to the maximum level of MMP13 secreted per cell from TNFα-treated, gel-coated MSCs (˜5 fg/cell). The conditioned medium from MMP13-containing microgels after release for 1 day showed an equivalent collagenase activity to the medium from TNFα-treated, gel-coated MSCs treated with 1 mM APMA. However, i.t. administration of 100,000 protein-loaded microgels per 20 g mice after 3 weeks of bleomycin treatment failed to reduce collagen deposition or immune cell infiltration in the airways. Thus, TNFα- or MMP13-loaded microgels alone were not sufficient to resolve fibrotic injury. Together, specifically defined chemomechanical cues enable MSCs to accelerate the resolution of fibrotic phenotypes as predicted by the mathematical model.

Success in some cell-based therapeutics depends on both a fundamental understanding of how macroenvironmental properties impact cellular functions and development of approaches to control these properties around donor cells. In the present invention, engineered gel coating was leveraged to specify chemomechanical cues for donor MSCs as a means to augment their ability to treat aberrant tissue remodeling after fibrotic injury. Specifically, experimental evidence demonstrates that cell therapy can be rationally designed by defining both soluble and insoluble signals, which together enhance the production of interstitial collagenases by MSCs. Conformal gel coating of individual MSCs can then be used to locally present these signals after tissue delivery. Mechanistic studies support a mathematical model that informs strategies to optimize the effect of MSCs when delivered in different stages of fibrotic injury.

While previous studies tested therapeutic effects of MSCs from a standard plastic culture in animal models of fibrotic lung injury, they showed variable outcomes (Srour & Thebaud (2015) Stem Cells Transl. Med. 4:1500). Only a couple of studies administered MSCs after at least 1 week (Ortiz, et al. (2003) Proc. Natl. Acad. Sci. USA 100:8407; Huang, et al. (2015) Mol. Med. Rep. 11:1685). Consistent with these studies, it has now been shown that MSCs without gel coating show limited efficacy when delivered 1 week after bleomycin treatment. The combined mathematical modeling and experimental approaches show that therapeutic efficacy of MSCs depends on a number of parameters, including the level of host TNFα, sensitivity of donor cells to TNFα ligand binding, and the maximum production rate of soluble interstitial collagenases, all of which may have contributed to variable outcomes from previous studies. Importantly, the results herein show that engineered gel coating can be used to optimize these parameters to improve MSC-based therapeutics against fibrotic lung injury.

The present study demonstrates that soft matrix potentiates the ability of MSCs to respond to TNFα, which results in increased downstream production of soluble interstitial collagenases by activating JNK. TNFα-induced JNK activation is known to be mediated by TNF receptor associated factor-2 (TRAF21; Reinhard, et al. (1997) EMBO J. 16:1080), which was isolated biochemically as a complex of TNF receptor 2 (TNFR2; Rothe, et al. (1994) Cell 78:681). Hydrogels present cells with cytokines in a matrix-bound form, thereby activating receptors that may be less potently engaged by ligands in a free-soluble form, such as TNFR2 (Grell, et al. (1995) Cell 83:793). Hydrogel stiffness can modulate TNFα-mediated activation of MSCs either by influencing ligand mobility (Huebsch, et al. (2010) Nat. Mater. 9:518), or regulating receptor activation or internalization (Du, et al. (2011) Proc. Natl. Acad. Sci. USA 108:9466). Miniaturizing a bulk gel into thin gel coating reduces material-to-cell volume ratios, which further influences cell membrane fluidity and mobility of receptors. Indeed, nascent protein production is enhanced in thin (5 μm) compared to thick (15 μm) hydrogel-coated MSCs. Notably, extracellular matrix gene expression (i.e., Klk8, Eno3, S100a16, G0s2, Fam19a5, Lamb3, Mustn1, Pgm5, Col5a3, and Col6a2) of MSCs is selectively increased in response to a thin (5 μm) cross-linked alginate hydrogel, but not to differences in hydrogel elasticity. These results highlight the importance of biomaterial design in sensitizing the activation of MSCs by a specific inflammatory signal to promote normal tissue remodeling as an adaptive response.

Cells possess tactile mechanisms that enable them to interpret properties of the surrounding matrix, and subsequently to control its turnover (Daley, et al. (2010) Nat. Med. 7:695). Gel coating presents MSCs with tactile matrix signals without a need for cells to cross endothelial or epithelial barriers to contact the extracellular matrix (ECM) in the interstitium, so that they can be poised to remodel the host matrix via paracrine secretions. This strategy is also suitable to control physical contact of donor cells with host tissues prior to repair or regeneration. To study the impact of specifically defined chemomechanical cues in vivo, gel formulation was analyzed to keep cells within gel coating, which were then cleared by the host without engraftment. Gel coating can further be modified to tune gel degradation (Khetan, et al. (2013) Nat. Mater. 12:458) or fast stress relaxation (Chaudhuri, et al. (2016) Nat. Mater. 15:326) properties, and to introduce additional chemical modifications for delayed clearance (Rodriguez, (2013) Science 339:971), followed by evaluation of their impact on both treating aberrant tissue remodeling and engraftment of donor cells to host tissues.

Bacterial collagenases have been clinically approved for Dupuytren's contracture (Hurst, et al. (2009) New Engl. J. Med. 361:968) and Peyronie's disease (Gelbard, et al. (2013) J. Urol. 190:199), but their safety and clinical utility in aberrant tissue remodeling remain unclear, thus highlighting the importance of studying novel tissue modeling therapies. Although it has been shown that the donor MMP13 and the host TNFα contribute to the ability of gel-coated MSCs to inhibit fibrotic injury, delivering catalytically-active MMP13 alone or together with TNFα via microgels alone was not sufficient to resolve fibrosis. These results suggest the possibility that resolution of fibrosis requires not only degradation of the fibrotic matrix but also restoration of a healthy matrix. Gel-coated MSCs may thus exert a dual function by degrading the fibrotic matrix via soluble collagenases and contributing to the restoration of the matrix, for example, by providing nascent matrix molecules. In addition, it is possible that more sustained degradation of the fibrotic matrix by controlled delivery of collagenases may resolve fibrosis. One approach to achieve this would be by sulfation of alginate gels to mimic heparin sulfate (Freeman, et al. (2008) Biomaterials 29:3260), which is known to bind to a number of MMP isoforms (Yu, et al. (2000) J. Biol. Chem. 275:418). In addition, understanding how MMPs from donor cells are delivered to the interstitium, as a function of routes of administration would refine the mathematical model to predict their efficacy in aberrant tissue remodeling of different organs.

Localization of Gel-Coated MSCs After Intramuscular Injection. To assess biodistribution of MSCs in live mice, firefly luciferase and mCherry-expressing marrow MSCs were used. Transduced MSCs were encapsulated in a thin (5 μm) alginate-RGD microgel and injected into the gastrocnemius muscle with an insulin syringe as described (Siemionow, et al. (2019) Stem Cell Rev. Rep. 15:827). This analysis showed that gel-coated MSCs could be delivered and remained localized within the injected gastrocnemius muscle after one day of delivery.

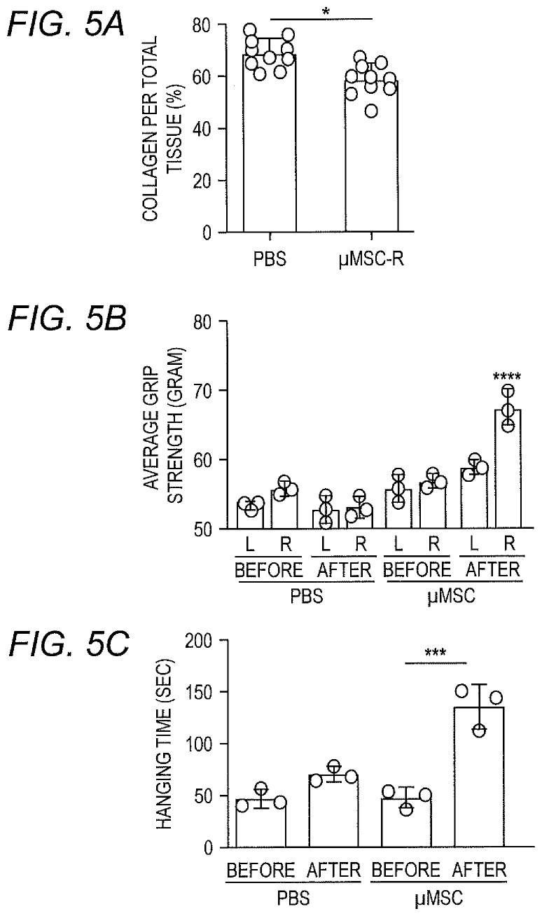

Gel-Coated MSCs can Reduce Collagen Deposition and Improve Muscle Functions in D2.mdx Mice. To investigate the therapeutic efficiency of engineered MSCs, thin (5 μm) gel-coated marrow MSCs (100,000/20 g mouse) were injected into the right leg of D2.mdx mice at 5 weeks of age, while the left leg received PBS as a control. After 2 weeks of the treatment, gel-coated MSCs reduced collagen deposition in skeletal muscles (FIG. 5A) and restored grip strength of the right leg compared to the left leg (FIG. 5B), indicating the localized effect of gel-coated MSCs. Notably, hanging ability of 4-limbs appeared to be improved by the treatment with gel-coated MSCs on the right leg alone (FIG. 5C). Thus, gel-coated MSCs can inhibit collagen deposition and improve muscle functions.

What is claimed is:

1. Alginate-coated cells comprising a cross-linked alginate hydrogel layer encapsulating single mesenchymal stromal cells or progenitor cells, wherein the alginate is conjugated to one or more cell adhesive ligands and the cross-linked alginate hydrogel layer has a thickness of less than about 10 microns.

2. The alginate-coated cells of claim 1, wherein the alginate hydrogel is cross-linked with a divalent or trivalent cation.

3. The alginate-coated cells of claim 1, wherein the cross-linked alginate hydrogel layer has a softness of about 0.1 to about 10 kPa.

4. The alginate-coated cells of claim 3, wherein the cross-linked alginate hydrogel layer has a softness of about 2 kPa.

5. The alginate-coated cells of claim 1, wherein the cross-linked alginate hydrogel layer has a thickness of about 0.5 to about 5 microns.

6. The alginate-coated cells of claim 1, wherein the cross-linked alginate hydrogel layer has a stress relaxation rate of about 4 seconds.

7. The alginate-coated cells of claim 1, wherein the alginate has a molecular weight of greater than about 250 kDa.

8. The alginate-coated cells of claim 7, wherein the alginate has a molecular weight of about 250 kDa to about 500 kDa.

9. The alginate-coated cells of claim 1, wherein the alginate hydrogel layer further comprises one or more growth factors, inflammatory factors, differentiation factors, or a combination thereof.

10. The alginate-coated cells of claim 9, wherein the growth factors comprise Bmp-2, Bmp2-derived agonist peptides, or BMP receptor agonists.

11. The alginate-coated cells of claim 9, wherein the inflammatory factors comprise TNFα, TNFα-derived agonist peptides, or TNF receptor agonists.

12. A composition comprising the alginate-coated cells of claim 1, and a pharmaceutically acceptable carrier or aqueous medium.

13. The composition of claim 12, further comprising one or more ion channel modulators, one or more cell contractility modulators, or a combination thereof.

14. A method for treating a subject comprising administering to a subject in need of treatment with mesenchymal stromal cells or progenitor cells an effective amount of the alginate-coated cells of claim 1 thereby treating the subject.

15. The method of claim 14, wherein the subject is in need of skin, bone, cartilage, muscle, lung, heart, kidney, or blood vessel tissue repair.

16. The method of claim 14, wherein the subject has lung fibrosis, muscle fibrosis, fibrosis of connective tissues, kidney fibrosis, liver fibrosis, corneal fibrosis, radiation-induced fibrosis, chronic graft versus host disease (GVHD)-induced fibrosis, systemic sclerosis, or myocardial infarction.

17. The method of claim 14, further comprising differentiating the alginate-coated cells prior to administering to the subject.

18. The method of claim 14, wherein the alginate-coated cells are administered by intratracheal instillation, intratracheal inhalation, intravenous delivery, intramuscular delivery, intraarterial delivery, topical delivery, renal artery injection, portal vein injection, intrabone delivery, intraarticular delivery, intralymphatic delivery, intrathymic delivery, intrarenal delivery, intracorneal delivery, intraportal delivery, intrahepatic delivery, or intracardiac injection.

19. A method of preparing a composition comprising a cross-linked alginate hydrogel layer encapsulating single mesenchymal stromal cells or progenitor cells comprising contacting an aqueous phase comprising mesenchymal stromal cells or progenitor cells and a divalent or trivalent cation with an oil phase comprising alginate conjugated to one or more cell adhesive ligands so that a cross-linked alginate hydrogel layer encapsulating single mesenchymal stromal cells or progenitor cells is formed, wherein the cross-linked alginate hydrogel layer has a thickness of less than about 10 microns and the hydrogel layer thickness can be modulated without changing gel viscoelasticity.

20. The method of claim 19, wherein the hydrogel layer has a thickness of 0.5 to about 5 microns.
